(19) United States
(12) Patent Application Publication
(10) Pub. No.: US 2022/0282316 A1
(43) Pub. Date: Sep. 8, 2022

(54) METHODS AND COMPOSITIONS FOR MODIFYING PRIMARY PROBES IN SITU

(71) Applicant: 10x Genomics, Inc., Pleasanton, CA (US)

(72) Inventor: Felice Alessio BAVA, Rome (IT)

(21) Appl. No.: 17/685,033

(22) Filed: Mar. 2, 2022

Related U.S. Application Data
(60) Provisional application No. 63/156,240, filed on Mar. 3, 2021.

(51) Int. Cl. C12Q 1/6837; C12Q 1/6874

(52) U.S. Cl. CPC C12Q 1/6837; C12Q 1/6874

(57) ABSTRACT

The present disclosure relates in some aspects to methods for analyzing a target nucleic acid in a biological sample. In some aspects, the methods involve the use of a set of oligonucleotides, for example a set of two or more oligonucleotides, wherein one or more oligonucleotides comprises modified nucleotides, for assessing target nucleic acids. In some aspects, the presence, amount, and/or identity of a target nucleic acid is analyzed in situ. Also provided are oligonucleotides, sets of oligonucleotides, compositions, and kits for use in accordance with the methods.

### CROSS-REFERENCE TO RELATED APPLICATIONS

This application claims priority to U.S. Provisional Patent Application No. 63/156,240, filed Mar. 3, 2021, entitled “METHODS AND COMPOSITIONS FOR MODIFYING PRIMARY PROBES IN SITU,” which is herein incorporated by reference in its entirety for all purposes.

### FIELD

The present disclosure relates in some aspects to methods and compositions for analysis of a target nucleic acid in a sample (e.g., in situ), such as analysis using modified (e.g., crosslinkable) probes.

### BACKGROUND

Oligonucleotide probe-based assay methods for analysis of target nucleic acids depend on careful optimization related to the stability of the hybridization complex and/or the positional stability of the hybridization complex. For example, if the wash conditions are too stringent, then probe/target hybrids will be denatured, resulting in a decrease in the amount of signal in the assay. Furthermore, some methods such as isometric expansion of a sample require stabilization of target analytes to a matrix in order to preserve positional information of the target analytes in the sample (e.g., a cell or tissue sample). Thus, there is a need for affordable and easily customizable probes comprising modified nucleotides (e.g., crosslinkable nucleotides) for use in analysis of target nucleic acids in a sample. Provided herein are methods and compositions that address such and other needs.

### BRIEF SUMMARY

In some aspects, provided herein is a method of modifying a probe, comprising: (a) contacting a probe, a first oligonucleotide, and a sample comprising a target nucleic acid in any suitable order, wherein: the probe comprises (i) a hybridization region that hybridizes to the target nucleic acid in the sample, (ii) a first overhang, and (iii) a second overhang, wherein the first and second overhangs do not hybridize to the target nucleic acid, and the second overhang hybridizes to the first oligonucleotide; and (b) attaching one or more modified nucleotides to the second overhang using the first oligonucleotide as a template or to a complement of the second overhang using the first oligonucleotide as a primer, thereby modifying the probe hybridized to the target nucleic acid in the sample.

In some embodiments, the second overhang is at the 3′ of the probe. In some embodiments, the attaching step comprises extending the 3′ of the second overhang.

In any of the preceding embodiments, the polymerase can catalyze extension of the second overhang using the first oligonucleotide as a template, thereby attaching the one or more modified nucleotides to the second overhang.

In any of the preceding embodiments, the polymerase may be a polymerase that does not have a strand displacing activity, e.g., a T4 or T7 polymerase.

In any of the preceding embodiments, the first oligonucleotide can be blocked at the 3′ from extension, e.g., primer extension catalyzed by a polymerase.

In any of the preceding embodiments, wherein the first oligonucleotide can comprise a 3′ modification. In some embodiments, the 3′ modification can be selected from the group consisting of 3′ ddC, 3′ inverted dT, a 3′ spacer phosphoramidite (e.g., a C3 spacer), 3′ amino, or a 3′ phosphorylation.

In any of the preceding embodiments, the extended second overhang can comprise two or more modified nucleotides.

In any of the preceding embodiments, the attaching step can comprise ligating the second overhang and a first extension oligonucleotide using the first oligonucleotide as a splint.

In any of the preceding embodiments, the first extension oligonucleotide can comprise two or more modified nucleotides.

In any of the preceding embodiments, the ligation may not be preceded by gap filling. In any of the preceding embodiments, the ligation may be preceded by gap filling. In some embodiments, the gap filling incorporates two or more modified nucleotides into the second overhang or the first extension oligonucleotide.

In any of the preceding embodiments, the ligation can be enzymatic ligation or chemical ligation, e.g., using click chemistry.

In any of the preceding embodiments, the second overhang can be at the 5′ of the probe. In some embodiments, the attaching step comprises extending the 5′ of the second overhang.

In any of the preceding embodiments, the attaching step can comprise ligating the second overhang and a first extension oligonucleotide using the first oligonucleotide as a splint. In some embodiments, the first extension oligonucleotide can comprise two or more modified nucleotides.

In any of the preceding embodiments, the ligation may not preceded by gap filling.

In any of the preceding embodiments, the ligation may be preceded by gap filling. In some embodiments, the gap filling incorporates two or more modified nucleotides into the first extension oligonucleotide.

In any of the preceding embodiments, the ligation can be enzymatic ligation or chemical ligation, e.g., using click chemistry.

In any of the preceding embodiments, the method can further comprise contacting the sample with a second oligonucleotide, wherein the second oligonucleotide hybridizes to a ligation product of the second overhang of the probe.

In any of the preceding embodiments, the method can comprise a step (c) of attaching one or more modified nucleotides to the ligation product of the second overhang using the second oligonucleotide as a template or into a complement of the ligation product of the second overhang using the second oligonucleotide as a primer, thereby modifying the probe hybridized to the target nucleic acid in the sample.

In some embodiments, the second overhang is at the 3′ of the probe and a polymerase can catalyze extension of the ligation product of the second overhang using the second oligonucleotide as a template, thereby attaching the one or more modified nucleotides to the second overhang. In other embodiments, the attaching in step (c) comprises ligating the ligation product of the second overhang and a second extension oligonucleotide using the second oligonucleotide as a splint.

In any of the preceding embodiments, the attaching step can comprise incorporating one or more modified nucleotides into the complement of the second overhang using the first oligonucleotide as a primer. In some embodiments, a polymerase can catalyze extension of the first oligonucleotide using the second overhang as a template, thereby incorporating the one or more modified nucleotides into the complement of the second overhang.

In any of the preceding embodiments, the first and/or second oligonucleotide can comprise one or more modified nucleotides.

In some aspects, provided herein is a method of modifying a probe, comprising: (a) contacting a probe, a first oligonucleotide, and a sample comprising a target nucleic acid in any suitable order, wherein: the probe comprises (i) a hybridization region that hybridizes to the target nucleic acid in the sample, (ii) a first overhang, and (iii) a second overhang, wherein the first and second overhangs do not hybridize to the target nucleic acid, and the second overhang hybridizes to the first oligonucleotide; and (b) ligating the second overhang to a first extension oligonucleotide comprising one or more modified nucleotides, using the first oligonucleotide as a template, thereby modifying the probe hybridized to the target nucleic acid in the sample.

In some aspects, provided herein is a method of modifying a probe, comprising: (a) contacting a probe, a first oligonucleotide, and a sample comprising a target nucleic acid in any suitable order, wherein: the probe comprises (i) a hybridization region that hybridizes to the target nucleic acid in the sample, (ii) a first overhang, and (iii) a second overhang at the 3′ end of the probe, wherein the first and second overhangs do not hybridize to the target nucleic acid, and the second overhang hybridizes to the first oligonucleotide; and (b) extending the second overhang or first oligonucleotide using a polymerase to incorporate one or more modified nucleotides to the second overhang using the first oligonucleotide as a template or into a complement of the second overhang using the first oligonucleotide as a primer, thereby modifying the probe hybridized to the target nucleic acid in the sample.

In some aspects, provided herein is a method of modifying a probe, comprising: (a) contacting a probe, a first oligonucleotide, and a sample comprising a target nucleic acid in any suitable order, wherein: the probe comprises (i) a hybridization region that hybridizes to the target nucleic acid in the sample, (ii) a first overhang, and (iii) a second overhang at the 3′ end of the probe, wherein the first and second overhangs do not hybridize to the target nucleic acid, and the second overhang hybridizes to the first oligonucleotide; and (b) extending the second overhang using a polymerase to incorporate one or more modified nucleotides to the second overhang using the first oligonucleotide as a template, thereby modifying the probe hybridized to the target nucleic acid in the sample; wherein the first oligonucleotide is a linear oligonucleotide. In some embodiments, the probe is not circular or circularized. In some embodiments, the first oligonucleotide is not circularized.

In any of the preceding embodiments, a duplex comprising the second overhang and the first oligonucleotide can be stabilized, e.g., via crosslinking strands of the duplex.

In any of the preceding embodiments, the one or more modified nucleotides can comprise one or more cross-linkable nucleotides. In some embodiments, the cross-linkable nucleotides comprise photo-crosslinkable nucleotides such as UV-crosslinkable nucleotides.

In any of the preceding embodiments, the one or more modified nucleotides can comprise a halogenated base, an azide-modified base, an octadiynyl dU, a thiol-modified base, a biotin-modified base, or a combination thereof.

In any of the preceding embodiments, the method can further comprise crosslinking the one or more modified nucleotides to the sample, a substrate, and/or a matrix, e.g., a hydrogel matrix, thereby crosslinking the probe to the sample, the substrate, and/or the matrix, thereby increasing positional stability of the probe relative to the sample. In some embodiments, the probe can be crosslinked to an endogenous molecule of the sample, e.g., an endogenous protein. In some embodiments, the sample is embedded in a matrix with functional moieties. In some embodiments, the method further comprises embedding the sample with a matrix with functional moieties prior to contacting the sample with a probe and a first oligonucleotide.

In any of the preceding embodiments, the one or more modified nucleotides can comprise at least one nucleotide that is internal after incorporation.

In any of the preceding embodiments, the one or more modified nucleotides can comprise a 3′ or 5′ terminal nucleotide after incorporation.

In any of the preceding embodiments, the one or more modified nucleotides comprise two or more different types of nucleotide modifications.

In any of the preceding embodiments, the first overhang can comprise one or more barcode sequences.

In any of the preceding embodiments, the first overhang can comprise one or more landing sequences capable of hybridizing to one or more secondary probes. In some embodiments, the one or more landing sequences are barcode sequences. In some embodiments, the one or more secondary probes can be detectably labeled.

In any of the preceding embodiments, the one or more secondary probes can comprise one or more adaptor sequences that do not hybridize to the landing sequence(s), wherein each adaptor sequence is capable of hybridizing to a detectably labeled oligonucleotide.

In any of the preceding embodiments, the sample can comprise cells, optionally wherein the sample is a processed or cleared biological sample. In some instances, the sample is embedded in a hydrogel.

In any of the preceding embodiments, the sample can be a tissue sample. In some embodiments, the sample is a tissue slice between about 1 μm and about 50 μm in thickness. In some embodiments, the tissue slice is between about 5 μm and about 35 μm in thickness.

In any of the preceding embodiments, the method can further comprise analyzing localization of the target nucleic acid in the sample.

In any of the preceding embodiments, the method can further comprise detecting a signal indicative of the probe hybridized to the target nucleic acid in the sample. In some embodiments, the detecting step can comprise in situ sequencing and/or in situ hybridization. In some embodiments, the in situ sequencing can comprise sequencing by ligation, sequencing by hybridization, sequencing by synthesis, and/or sequencing by binding. In some embodiments, the in situ hybridization can comprise sequential fluorescent in situ hybridization.

In any of the preceding embodiments, the attaching step can be performed after contacting the sample comprising the target nucleic acid with the probe and the first oligonucleotide. In some embodiments, the attaching step can performed after the probe is hybridized to the target nucleic acid.

In any of the preceding embodiments, the target nucleic acid can be a viral or cellular DNA or RNA. In any of the preceding embodiments, the target nucleic acid comprises genomic DNA/RNA, mRNA, or cDNA.

In any of the preceding embodiments, the target nucleic acid can be endogenous in the sample.

In any of the preceding embodiments, the target nucleic acid in the sample can be a product of an endogenous molecule in the sample. In some embodiments, the product comprises a hybridization product, a ligation product, an extension product (e.g., by a DNA or RNA polymerase), a replication product, a transcription/reverse transcription product, and/or an amplification product such as a rolling circle amplification (RCA) product of an endogenous molecule in the sample.

In any of the preceding embodiments, the target nucleic acid in the sample can be comprised in a labelling agent that directly or indirectly binds to an analyte in the sample, or can be comprised in a product (e.g., a hybridization product, a ligation product, an extension product (e.g., by a DNA or RNA polymerase), a replication product, a transcription/reverse transcription product, and/or an amplification product such as a rolling circle amplification (RCA) product) of the labelling agent. In some embodiments, the labelling agent can comprise a reporter oligonucleotide. In some instances, the reporter oligonucleotide comprises one or more barcode sequences and the product of the labelling agent comprises one or a plurality of copies of the one or more barcode sequences.

In any of the preceding embodiments, the target nucleic acid in the sample can be a rolling circle amplification (RCA) product of a circular or circularizable (e.g., padlock) probe or probe set that hybridizes to a DNA (e.g., a cDNA of an mRNA) or RNA (e.g., an mRNA) molecule in the sample.

In any of the preceding embodiments, the labelling agent can comprise a binding moiety that directly or indirectly binds to a non-nucleic acid analyte in the sample, e.g., an analyte comprising a peptide, a protein, a carbohydrate, and/or lipid, and the reporter oligonucleotide in the labelling agent identifies the binding moiety and/or the non-nucleic acid analyte.

In any of the preceding embodiments, the binding moiety of the labelling agent can comprise an antibody or antigen binding fragment thereof that directly or indirectly binds to a protein analyte, and the nucleic acid molecule in the sample can be a rolling circle amplification (RCA) product of a circular or circularizable (e.g., padlock) probe or probe set that hybridizes to a reporter oligonucleotide of the labelling agent.

### BRIEF DESCRIPTION OF THE DRAWINGS

The drawings illustrate certain embodiments of the features and advantages of this disclosure. These embodiments are not intended to limit the scope of the appended claims in any manner.

FIGS. 1A-1B show an exemplary method of modifying a probe by extension and incorporation of modified nucleotides using a first oligonucleotide as a template. As shown in FIG. 1A, the probe comprises (i) a hybridization region that hybridizes to the target nucleic acid in the sample, (ii) a first overhang, and (iii) a second overhang, wherein the first and second overhangs do not hybridize to the target nucleic acid. In some embodiments, the second overhang can be at the 3′ end of the probe, as shown. The first oligonucleotide hybridizes to the second overhang, providing a template for extension of the probe using a polymerase to incorporate one or more modified nucleotides, and using the first oligonucleotide as a template (FIG. 1B). In some embodiments, the first overhang can comprise one or more barcode sequences.

FIGS. 2A-2B show an exemplary method of modifying a probe by attaching an extension oligonucleotide comprising one or more modified nucleotides to the second overhang by ligation, wherein a first oligonucleotide acts as a splint to template the ligation. As shown in FIG. 2A, the second overhang can be located at either the 5′ end or the 3′ end of the probe. The sample is contacted with a first extension oligonucleotide comprising one or more modified nucleotides and a first oligonucleotide, wherein the first oligonucleotide hybridizes to the second overhang. In some embodiments, the first extension oligonucleotide can extend beyond the first oligonucleotide (i.e., can comprise a region that does not hybridize to the first oligonucleotide), as shown in FIG. 2B.

FIG. 3A shows an exemplary method of modifying a probe by attaching a first and a second extension oligonucleotide, wherein the first extension oligonucleotide is ligated to the second overhang using a first oligonucleotide as a splint (i.e., as a template for ligation), and the second extension oligonucleotide is ligated to the ligation product of the second overhang using a second oligonucleotide as a splint. The first and/or the second extension oligonucleotide can comprise one or more modified nucleotides.

FIG. 3B shows an exemplary method of modifying a probe by attaching a first extension oligonucleotide to the second overhang by ligation using a first oligonucleotide as a splint, and extending the ligation product of the second overhang using a second oligonucleotide as a template. The second oligonucleotide hybridizes to the extended second overhang, providing a template for extension of the probe using a polymerase to incorporate one or more modified nucleotides.

FIGS. 4A-4B show an exemplary method of modifying a probe by attaching one or more modified nucleotides to the second overhang of the probe, wherein said modified nucleotides are incorporated into a complement of the second overhang using a first oligonucleotide as a primer and the second overhang as a template for extension by a polymerase. In this example, the modified oligonucleotides are indirectly attached to the probe by hybridization of the modified extended first oligonucleotide and the second overhang. In some embodiments, the second overhang is at the 5′ end of the probe and the first oligonucleotide hybridizes at the 3′ end of the second overhang (FIG. 4A). In other embodiments, the second overhang is at the 3′ end of the probe and the first oligonucleotide hybridizes at the 3′ end of the second overhang (FIG. 4B). In some embodiments of the method shown in FIG. 4B, the polymerase does not have strand-displacing activity or hybridization of a xenonucleic acid (XNA) to the 5′ end of the second overhang can block extension beyond the 5′ end of the second overhang, thus blocking displacement of the probe from the target nucleic acid.

FIG. 5A shows an exemplary method wherein the one or more modified nucleotides comprise one or more cross-linkable nucleotides. Cross-linking is indicated by an “x”. In some embodiments, the methods provided herein allow incorporation of multiple crosslinkable nucleotides into the probe. In some embodiments, the method comprises crosslinking the one or more modified nucleotides to the sample, a substrate, and/or a matrix, e.g., a hydrogel matrix, thereby crosslinking the probe to the sample, the substrate, and/or the matrix, thereby increasing positional stability of the probe relative to the sample. Ins some embodiments, the probe is crosslinked to an endogenous molecule of the sample, e.g., an endogenous protein.

FIG. 5B shows an exemplary method of detecting a modified probe by hybridization of one or more secondary probes to the first overhang of the probe. In some embodiments, the first overhang can comprise one or more barcode sequences. In some embodiments, the first overhang can comprise one or more landing sequences capable of hybridizing to one or more secondary probes, optionally wherein the one or more landing sequences are barcode sequences. The one or more secondary probes can be detectably labeled, or can comprise one or more adaptor sequences that do not hybridize to the landing sequence(s), wherein each adaptor sequence is capable of hybridizing to one or more detectably labeled oligonucleotides, as shown in FIG. 5B. It will be understood that the detection methods are not limited to the example shown, and that any suitable method can be used to detect the probe, including for example sequential hybridization, sequencing by hybridization, sequencing by ligation, sequencing by synthesis, sequencing by binding, hybridization chain reaction, or any combination thereof.

### DETAILED DESCRIPTION

The practice of the techniques described herein may employ, unless otherwise indicated, conventional techniques and descriptions of organic chemistry, polymer technology, molecular biology (comprising recombinant techniques), cell biology, biochemistry, and sequencing technology, which are within the skill of those who practice in the art. Such conventional techniques comprise polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W. H. Freeman, New York N.Y.; Gait, “Oligonucleotide Synthesis: A Practical Approach” 1984, IRL Press, London; Nelson and Cox (2000), Lehninger, Principles of Biochemistry 3rd Ed., W. H. Freeman Pub., New York, N.Y.; and Berg et al. (2002) Biochemistry, 5^{th} Ed., W. H. Freeman Pub., New York, N.Y., all of which are herein incorporated in their entirety by reference for all purposes.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods involving the use of a set of polynucleotides for modifying a probe used for analyzing one or more target nucleic acid(s), such as a target nucleic acid (for example, a messenger RNA or analyte comprising a nucleic acid) present in a sample (e.g. cell or a biological sample, such as a tissue sample). Also provided are polynucleotides, sets of polynucleotides, compositions, kits, systems and devices for use in accordance with the provided methods. In some aspects, the provided methods can be applied to introduce one or more (e.g., two or more) modified nucleotides, such as crosslinkable nucleotides, into a probe for analysis of a target nucleic acid.

In some aspects, provided herein are methods of modifying a probe, comprising: (a) contacting a probe, a first oligonucleotide, and a sample comprising a target nucleic acid in any suitable order, wherein: the probe comprises (i) a hybridization region that hybridizes to the target nucleic acid in the sample, (ii) a first overhang, and (iii) a second overhang, wherein the first and second overhangs do not hybridize to the target nucleic acid, and the second overhang hybridizes to the first oligonucleotide; and (b) attaching one or more modified nucleotides to the second overhang using the first oligonucleotide as a template or into a complement of the second overhang using the first oligonucleotide as a primer, thereby modifying the probe hybridized to the target nucleic acid in the sample. In some embodiments, the second overhang is at the 3′ end of the probe. In some embodiments, the second overhang is at the 5′ end of the probe.

In some aspects, provided herein are methods of modifying a probe comprising extending a second overhang of the probe using a polymerase to attach one or more modified nucleotides, using a first oligonucleotide as a template. In some embodiments, the second overhang is at the 3′ of the probe. In some embodiments, the attaching step comprises extending the 3′ of the second overhang. In some embodiments, the polymerase does not have a strand displacing activity, e.g., the polymerase is a T4 or T7 polymerase. This can prevent extension of the 3′ end of the first oligonucleotide from displacing the probe from the target nucleic acid. In some embodiments, the first oligonucleotide is blocked at the 3′ from extension, e.g., primer extension catalyzed by a polymerase. In some embodiments, the first oligonucleotide comprises a 3′ modification (e.g., a modification that blocks extension by a polymerase). Exemplary 3′ modifications include but are not limited to a 3′ ddC, 3′ inverted dT, a 3′ spacer phosphoramidite (e.g., a C3 spacer), 3′ amino, or a 3′ phosphorylation. In some embodiments, the extended second overhang comprises two or more modified nucleotides. In some embodiments, the two or more modified nucleotides can comprise the same modifications or different modifications.

In some aspects, provided herein are methods of modifying a probe comprising attaching one or more modified nucleotides to a second overhang of the probe, wherein the attaching step comprises ligating the second overhang and a first extension oligonucleotide using a first oligonucleotide as a splint, wherein the first extension oligonucleotide comprises one or more modified nucleotides. In some embodiments, the second overhang is at the 3′ end of the probe. In some embodiments, the second overhang is at the 5′ end of the probe. In some embodiments, the first extension oligonucleotide comprises two or more modified nucleotides. In some embodiments, the ligation is not preceded by gap filling. In some embodiments, the ligation is preceded by gap filling, optionally wherein the gap filling incorporates two or more modified nucleotides into the second overhang or into the extension oligonucleotide prior to ligation. In some embodiments, the ligation is enzymatic ligation or chemical ligation, e.g., using click chemistry.

In some aspects, the methods provided herein further comprise contacting the sample with a second oligonucleotide, wherein the second oligonucleotide hybridizes to a ligation product of the second overhang. In some embodiments, the method comprises attaching one or more modified nucleotides to the ligation product of the second overhang using the second oligonucleotide as a template or into a complement of the ligation product of the second overhang using the second oligonucleotide as a primer, thereby modifying the probe hybridized to the target nucleic acid in the sample. In some embodiments, the second overhang is at the 3′ of the probe and the second attaching step comprises extending the 3′ of the ligation product of the second overhang. In some embodiments, the second overhang is at the 5′ or 3′ end of the probe and the second attaching step comprises ligating the end of the ligation product of the second overhang and a second extension oligonucleotide using the second oligonucleotide as a splint.

In some aspects of the methods provided herein, the one or more modified nucleotides attached to the probe can be attached via hybridization of the second overhang to one or more probes comprising modified nucleotides. In some aspects, attaching one or more modified nucleotides to the probe comprises directly attaching (e.g., via ligation of an oligonucleotide or incorporation of modified nucleotides using a polymerase) one or more modified nucleotides to an oligonucleotide that is hybridized to the probe. For example, in some embodiments, the attaching step comprises incorporating one or more modified nucleotides into the complement of the second overhang using the oligonucleotide as a primer. In some embodiments, a polymerase catalyzes extension of the oligonucleotide using the second overhang as a template, thereby incorporating the one or more modified nucleotides into the complement of the second overhang. In some embodiments, the second overhang can first be extended using a first oligonucleotide as a splint (e.g., by ligating a first extension oligonucleotide comprising one or more modified nucleotides to the second overhang), and the first oligonucleotide can then one or more modified nucleotides can be incorporated into the complement of the second overhang using the first oligonucleotide as a primer. In some embodiments, the first and/or second oligonucleotide comprises one or more modified nucleotides. In some embodiments, a duplex comprising the second overhang and the first oligonucleotide or a duplex comprising the complement of the second overhang and the oligonucleotide can be stabilized, e.g., via crosslinking strands of the duplex.

In some aspects, the methods provided herein comprise attachment of one or more modified nucleotides, such as cross-linkable nucleotides. In a non-limiting example, the one or more modified nucleotides comprise one or more cross-linkable nucleotides, e.g., photo-crosslinkable nucleotides such as UV-crosslinkable nucleotides. In some embodiments, the one or more modified nucleotides comprise a halogenated base, an azide-modified base, an octadiynyl dU, a thiol-modified base, a biotin-modified base, or a combination thereof. In some embodiments, the one or more modified nucleotides comprise nucleotides compatible with specific attachment to another molecule (e.g., attachment of a biotin-modified nucleotide to a labelling agent or analyte comprising a streptavidin label, or attachment, or attachment using click chemistry). In some embodiments, the one or more modified nucleotides comprise nucleotides capable of reversible crosslinking. For example, a thiol-modified base may form a disulfide bond with a thiol group, such that if the disulfide bond is broken (e.g., in the presence of a reducing agent), the cross-linked agent is released from the probe. In other cases, the modified base a reactive hydroxyl group that may be used for attachment. In some embodiments, the one or more modified nucleotides comprise at least one nucleotide that is internal after incorporation. In some embodiments, the one or more modified nucleotides comprise a 3′ or 5′ terminal nucleotide after incorporation.

In some aspects, the methods provided herein comprise incorporation or attachment of two or more (e.g., 3 or more, 4 or more, 5 or more, 6 or more, 7 or more, 8 or more, 9 or more, or 10 or more) modified nucleotides to the probe. In some embodiments, the two or more modified nucleotides can comprise the same modifications or different modifications. In some embodiments, the two or more modified nucleotides can comprise different modifications having different functionalities (e.g., specific cross-linking or attachment to other agents vs. and non-specific cross-linking; or reversible cross-linking and irreversible cross-linking). The inclusion of multiple modified nucleotides in the probe may enable attachment to multiple different agents (e.g., attachment to a matrix and/or attachment to an endogenous protein or a specifically labeled agent), and/or may improve efficiency of cross-linking as each probe can comprise multiple cross-linkable nucleotides.

In some aspects, the methods provided herein comprise crosslinking the one or more modified nucleotides to the sample, a substrate, and/or a matrix, e.g., a hydrogel matrix, thereby crosslinking the probe to the sample, the substrate, and/or the matrix. In some cases, the crosslinking can increase positional stability of the probe relative to the sample and keep the probe in place in the sample (e.g., maintain positional information of the probe and associated target nucleic acid in the sample). In some aspects, the methods provided herein comprise crosslinking the one or more modified nucleotides of a first strand of a duplex (e.g., the duplex comprising the second overhang hybridized to the first oligonucleotide), to the second strand of the duplex, thereby stabilizing the duplex. In some aspects, the methods provided herein comprise crosslinking the probe to an endogenous molecule of the sample, e.g., an endogenous protein.

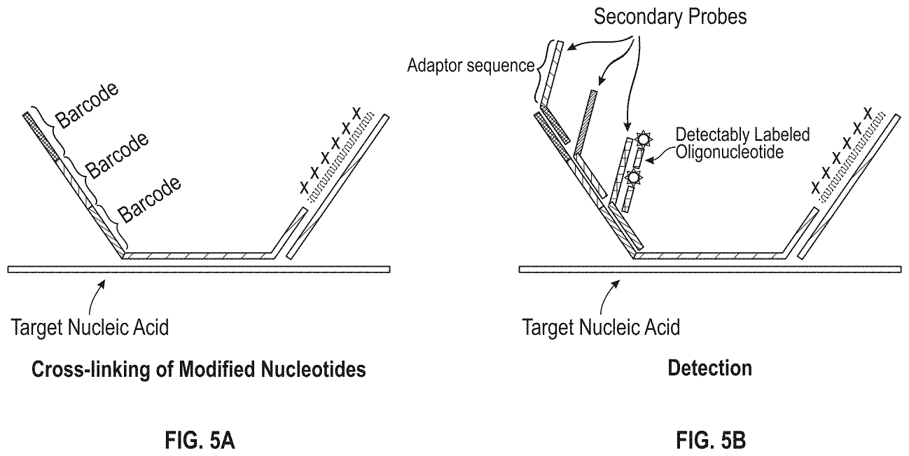

In some aspects, the probes provided herein comprise a first overhang, wherein the first overhang comprises one or more sequences used for detection of the probe (e.g., by hybridization of secondary probes or detection probes (e.g., detectably labeled oligonucleotides or secondary probes that comprise an adaptor sequence for hybridization of additional probes). In some embodiments, the first overhang comprises one or more barcode sequences. In some embodiments, the first overhang comprises one or more landing sequences capable of hybridizing to one or more secondary probes, optionally wherein the one or more landing sequences are barcode sequences. In some embodiments, the one or more secondary probes are detectably labeled. In some embodiments, the one or more secondary probes comprise one or more adaptor sequences that do not hybridize to the landing sequence(s), wherein each adaptor sequence is capable of hybridizing to a detectably labeled oligonucleotide (e.g., as shown in FIG. 5B).

In some embodiments, provided herein is a method of modifying a probe after it has been contacted with a sample (e.g., modifying a probe that is already hybridized to a target nucleic acid in the sample). In some embodiments, the sample comprises cells, optionally wherein the sample is a processed or cleared biological sample optionally embedded in a hydrogel. In some embodiments, the sample is a tissue sample, optionally a tissue slice between about 1 μm and about 50 μm in thickness, optionally wherein the tissue slice is between about 5 μm and about 35 μm in thickness. In some embodiments, the method further comprises analyzing localization of the target nucleic acid in the sample. In some embodiments, the one or more modified nucleotides are crosslinked to the sample, a substrate, and/or a matrix, e.g., a hydrogel matrix, thereby crosslinking the probe to the sample, the substrate, and/or the matrix, thereby increasing positional stability of the probe relative to the sample prior to detecting the signal indicative of the probe hybridized to the target nucleic acid in the sample. In some embodiments, the method further comprises detecting a signal indicative of the probe hybridized to the target nucleic acid in the sample.

In some aspects, the methods provided herein enable analysis of a target nucleic acid. In some embodiments, the target nucleic acid is a viral or cellular DNA or RNA, such as genomic DNA/RNA, mRNA, or cDNA. In some embodiments, the target nucleic acid is endogenous in the sample. In some embodiments, the target nucleic acid in the sample is a product (e.g., a hybridization product, a ligation product, an extension product (e.g., by a DNA or RNA polymerase), a replication product, a transcription/reverse transcription product, and/or an amplification product such as a rolling circle amplification (RCA) product) of an endogenous molecule in the sample. In some embodiments, the target nucleic acid in the sample is comprised in or by a labelling agent that directly or indirectly binds to an analyte in the sample (e.g., a reporter oligonucleotide of a labelling agent), or is comprised in a product (e.g., a hybridization product, a ligation product, an extension product (e.g., by a DNA or RNA polymerase), a replication product, a transcription/reverse transcription product, and/or an amplification product such as a rolling circle amplification (RCA) product) of the labelling agent. In some embodiments, the labelling agent comprises a reporter oligonucleotide, optionally wherein the reporter oligonucleotide comprises one or more barcode sequences and the product of the labelling agent comprises one or a plurality of copies of the one or more barcode sequences. In some embodiments, the target nucleic acid in the sample is a rolling circle amplification (RCA) product of a circular or circularizable (e.g., padlock) probe or probe set that hybridizes to a DNA (e.g., a cDNA of an mRNA) or RNA (e.g., an mRNA) molecule in the sample. In some embodiments, the labelling agent comprises a binding moiety that directly or indirectly binds to a non-nucleic acid analyte in the sample, e.g., an analyte comprising a peptide, a protein, a carbohydrate, and/or lipid, and the reporter oligonucleotide in the labelling agent identifies the binding moiety and/or the non-nucleic acid analyte. In some embodiments, the binding moiety of the labelling agent comprises an antibody or antigen binding fragment thereof that directly or indirectly binds to a protein analyte, and the nucleic acid molecule in the sample is a rolling circle amplification (RCA) product of a circular or circularizable (e.g., padlock) probe or probe set that hybridizes to a reporter oligonucleotide of the labelling agent. In some embodiments, the method does not comprise generating and/or detecting an amplification product (e.g., RCA product). In some aspects, provided herein is a method for modifying a probe hybridized to a target nucleic acid where amplification is not performed and the probe itself can be attached to a matrix or other components of the sample. In some cases, the probe itself being crosslinked allows positional information (e.g., localization in the sample) of the probe and its associated target nucleic acid to be retained.

In some embodiments, the probe is detected by in situ sequencing and/or in situ hybridization (e.g., sequencing of one or more barcodes comprised by the first overhang. In some embodiments, the in situ sequencing comprises sequencing by ligation, sequencing by hybridization, sequencing by synthesis, and/or sequencing by binding. In some embodiments, the in situ hybridization comprises sequential fluorescent in situ hybridization.

In some embodiments, provided herein is a method of modifying a probe comprising: (a) contacting a probe, a first oligonucleotide, and a sample comprising a target nucleic acid in any suitable order, wherein: the probe comprises (i) a hybridization region that hybridizes to the target nucleic acid in the sample, (ii) a first overhang, and (iii) a second overhang, wherein the first and second overhangs do not hybridize to the target nucleic acid, and the second overhang hybridizes to the first oligonucleotide; and (b) ligating the second overhang to a first extension oligonucleotide comprising one or more modified nucleotides, using the first oligonucleotide as a template, thereby modifying the probe hybridized to the target nucleic acid in the sample.

In some embodiments, provided herein is a method of modifying a probe, comprising: (a) contacting a probe, a first oligonucleotide, and a sample comprising a target nucleic acid in any suitable order, wherein: the probe comprises (i) a hybridization region that hybridizes to the target nucleic acid in the sample, (ii) a first overhang, and (iii) a second overhang at the 3′ end of the probe, wherein the first and second overhangs do not hybridize to the target nucleic acid, and the second overhang hybridizes to the first oligonucleotide; and (b) extending the second overhang or first oligonucleotide using a polymerase to incorporate one or more modified nucleotides to the second overhang using the oligonucleotide as a template or into a complement of the second overhang using the first oligonucleotide as a primer, thereby modifying the probe hybridized to the target nucleic acid in the sample.

In some embodiments, provided herein is method of modifying a probe, comprising: (a) contacting a probe, a first oligonucleotide, and a sample comprising a target nucleic acid in any suitable order, wherein: the probe comprises (i) a hybridization region that hybridizes to the target nucleic acid in the sample, (ii) a first overhang, and (iii) a second overhang at the 3′ end of the probe, wherein the first and second overhangs do not hybridize to the target nucleic acid, and the second overhang hybridizes to the first oligonucleotide; and (b) extending the second overhang using a polymerase to incorporate one or more modified nucleotides to the second overhang using the first oligonucleotide as a template, thereby modifying the probe hybridized to the target nucleic acid in the sample; wherein the first oligonucleotide is a linear oligonucleotide. In some embodiments, the probe is not circular or circularized. In some embodiments, the first oligonucleotide is not circularized.

### II. Samples, Analytes, and Target Sequences

A method disclosed herein may be used to process and/or analyze any analyte(s) of interest, for example, for detecting the analyte(s) in situ in a sample of interest. A target nucleic acid sequence for a probe modified by the methods disclosed herein may be or be comprised in an analyte (e.g., a nucleic acid analyte, such as genomic DNA, mRNA transcript, or cDNA, or a product thereof, e.g., an extension or amplification product, such as an RCA product) and/or may be or be comprised in a labelling agent for one or more analytes (e.g., a nucleic acid analyte or a non-nucleic acid analyte) in a sample or a product of the labelling agent. Exemplary analytes and labelling agents are described below. In some embodiments, the target nucleic acid sequence is in an amplification product formed using isothermal amplification or non-isothermal amplification, optionally rolling circle amplification (RCA). In some embodiments, the target nucleic acid sequence is in a probe or probe set that targets the amplification product. In some embodiments, the target nucleic acid sequence comprises a barcode sequence corresponding to an analyte.

### A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include tissues, cells, and/or molecules on a solid support, and can include a two-dimensional (2D) surface or a three-dimensional (3D) matrix. In some embodiments, analytes (e.g., protein, RNA, and/or DNA) can be provided on a 2D surface. In some embodiments, a 2D array comprises amplicons (e.g., rolling circle amplification products) derived from analytes (e.g., protein, RNA, and/or DNA) on a 2D surface. In some embodiments, a 2D surface may comprise a glass, plastic, or metal surface, optionally coated with a polymer, particle, protein, or combination thereof. In some embodiments, analytes (e.g., protein, RNA, and/or DNA) can be provided in a 3D matrix. In some embodiments, a 3D array comprises amplicons (e.g., rolling circle amplification products) derived from analytes (e.g., protein, RNA, and/or DNA) in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In general, the embedding material is removed prior to analysis of tissue sections obtained from the sample. Suitable embedding materials include, but are not limited to, waxes, resins (e.g., methacrylate resins), epoxies, and agar.

In some embodiments, the biological sample can be embedded in a hydrogel matrix. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method known in the art.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample are known in the art, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a hydrogel can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. In some embodiments, one or more modified nucleotides as described in section VI can be crosslinked to a matrix (e.g., a gel), thereby anchoring the probe to the matrix, followed by gel formation, proteolysis, and swelling. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, Mass.), Label-IT Amine (available from MirusBio, Madison, Wis.) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2x, 2.1x, 2.2x, 2.3x, 2.4x, 2.5x, 2.6x, 2.7x, 2.8x, 2.9x, 3x, 3.1x, 3.2x, 3.3x, 3.4x, 3.5x, 3.6x, 3.7x, 3.8x, 3.9x, 4x, 4.1x, 4.2x, 4.3x, 4.4x, 4.5x, 4.6x, 4.7x, 4.8x, or 4.9x its non-expanded size. In some embodiments, the sample is isometrically expanded to at least 2x and less than 20x of its non-expanded size.

(vii) Crosslinking and De-Crosslinking

In some embodiments, the biological sample is reversibly cross-linked prior to or during an in situ assay. In some embodiments, the biological sample can be cross-linked one or more times to anchor various components of the sample to the matrix. In some aspects, the polynucleotides and/or a derivative associated with an analyte or a probe bound thereto can be anchored to a polymer matrix. For example, the polymer matrix can be a hydrogel. In some embodiments, one or more of the polynucleotide probe(s) and/or a derivative thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules. In some embodiments, a labelling agent that directly or indirectly binds to an analyte in the sample comprises a reporter oligonucleotide and the reporter oligonucleotide may be cross-linked to the matrix.

In some embodiments, the biological sample is immobilized in a hydrogel via cross-linking of the polymer material that forms the hydrogel. Cross-linking can be performed chemically and/or photochemically, or alternatively by any other hydrogel-formation method known in the art. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. Non-chemical permeabilization methods are known in the art. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some embodiments, one or more nucleic acid probes can be used to hybridize to a target nucleic acid (e.g., cDNA or RNA molecule, such as an mRNA) and ligated in a templated ligation reaction (e.g., RNA-templated ligation (RTL) or DNA-templated ligation (e.g., on cDNA)) to generate a product for analysis. In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of captured RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods known to the field (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V.A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

### B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or 0-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence. In some embodiments, the analytes comprises one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

### (ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may include an agent that interacts with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a pmagnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (i.e., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labelling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labelling Agent

In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product) or derivative thereof is analyzed. In some embodiments, a labelling agent (or a reporter oligonucleotide attached thereto) that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product) or derivative of a labelling agent that directly or indirectly binds to an analyte in the biological sample is analyzed. Provided herein are methods involving the use of a set of polynucleotides for modifying a probe used for analyzing one or more target nucleic acid(s), such as a reporter oligonucleotide attached to a labelling agent contacted with a sample, wherein the methods comprise attachment of one or more modified nucleotides, such as cross-linkable nucleotides.

a. Hybridization

In some embodiments, a product of an endogenous analyte and/or a labelling agent is a hybridization product comprising the pairing of substantially complementary or complementary nucleic acid sequences within two different molecules, one of which is the endogenous analyte or the labelling agent. The other molecule can be another endogenous molecule or another labelling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labelling agent and each probe may comprise one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary. In some cases, the probe or probe sets used for analyzing a reporter oligonucleotide attached to a labelling agent can be modified by attaching one or more modified nucleotides, such as cross-linkable nucleotides.

b. Ligation

In some embodiments, a product of an endogenous analyte and/or a labelling agent is a ligation product that can be detected by any of the probes provided herein. In some embodiments, the ligation product is formed between two or more endogenous analytes. In some embodiments, the ligation product is formed between an endogenous analyte and a labelling agent. In some embodiments, the ligation product is formed between two or more labelling agent. In some embodiments, the ligation product is an intramolecular ligation of an endogenous analyte. In some embodiments, the ligation product is an intramolecular ligation of a labelling agent, for example, the circularization of a circularizable probe or probe set upon hybridization to a target sequence. The target sequence can be comprised in an endogenous analyte (e.g., genomic DNA or mRNA) or a product thereof (e.g., cDNA from a cellular mRNA transcript), or in a labelling agent (e.g., the reporter oligonucleotide) or a product thereof

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, a probe such as a padlock probe may be used to analyze a reporter oligonucleotide, which may generated using proximity ligation or be subjected to proximity ligation. In some examples, the reporter oligonucleotide of a labelling agent that specifically recognizes a protein can be analyzed using in situ hybridization (e.g., sequential hybridization) and/or in situ sequencing. Further, the reporter oligonucleotide of the labelling agent and/or a complement thereof and/or a product (e.g., a hybridization product, a ligation product, an extension product (e.g., by a DNA or RNA polymerase), a replication product, a transcription/reverse transcription product, and/or an amplification product) thereof can be recognized by another labelling agent and analyzed.

In some embodiments, an analyte (a nucleic acid analyte or non-nucleic acid analyte) can be specifically bound by two labelling agents (e.g., antibodies) each of which is attached to a reporter oligonucleotide (e.g., DNA) that can participate in ligation, replication, and sequence decoding reactions, e.g., using a probe or probe set. In some embodiments, the probe set may comprise two or more probe oligonucleotides, each comprising a region that is complementary to each other. For example, a proximity ligation reaction can include reporter oligonucleotides attached to pairs of antibodies that can be joined by ligation if the antibodies have been brought in proximity to each other, e.g., by binding the same target protein (complex), and the DNA ligation products that form are then used to template PCR amplification, as described for example in Soderberg et al., Methods. (2008), 45(3): 227-32, the entire contents of which are incorporated herein by reference. In some embodiments, a proximity ligation reaction can include reporter oligonucleotides attached to antibodies that each bind to one member of a binding pair or complex, for example, for analyzing a binding between members of the binding pair or complex. For detection of analytes using oligonucleotides in proximity, see, e.g., U.S. Patent Application Publication No. 2002/0051986, the entire contents of which are incorporated herein by reference. In some embodiments, two analytes in proximity can be specifically bound by two labelling agents (e.g., antibodies) each of which is attached to a reporter oligonucleotide (e.g., DNA) that can participate, when in proximity when bound to their respective targets, in ligation, replication, and/or sequence decoding reactions

In some embodiments, one or more analytes can be specifically bound by two primary antibodies, each of which is in turn recognized by a secondary antibody each attached to a reporter oligonucleotide (e.g., DNA). Each nucleic acid molecule can aid in the ligation of the probe to form a circularized probe. In some instances, the probe can comprise one or more barcode sequences that can be analyzed using any suitable method disclosed herein for in situ analysis.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

c. Primer Extension and Amplification

In some embodiments, a product here is a primer extension product of an analyte, a labelling agent, a probe or probe set bound to the analyte, or a probe or probe set bound to the labelling agent. In some embodiments, a product can be contacted with a probe and a set of polynucleotides for modifying the probe by attachment of one or more modified nucleotides, such as cross-linkable nucleotides.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (i.e., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labelling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) are known in the art such as linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1 :1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, US 2016/0024555, US 2018/0251833, US 2016/0024555, US 2018/0251833 and US 2017/0219465, each of which is herein incorporated by reference in its entirety. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. As noted above, many assays are known for the detection of numerous different analytes, which use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (i.e. a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein (e.g., a probe comprising a second overhang for attachment of one or more modified nucleotides) may be a hybridization complex formed of a cellular nucleic acid in a sample and an exogenously added nucleic acid probe. The exogenously added nucleic acid probe may be optionally ligated to a cellular nucleic acid molecule or another exogenous nucleic acid molecule. In other examples, a product comprising a target sequence for a probe disclosed herein (e.g., a probe comprising a second overhang for attachment of one or more modified nucleotides) may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a probe disclosed herein (e.g., a probe comprising a second overhang for attachment of one or more modified nucleotides) may be a probe hybridizing to an RCP. The probe may comprise an overhang that does not hybridize to the RCP but hybridizes to another probe (e.g., a probe comprising a second overhang for attachment of one or more modified nucleotides).

### C. Target Sequences

A target sequence for a probe disclosed herein (e.g., a probe that can be modified by any of the methods described herein) may be comprised in any analyte disclose herein, including an endogenous analyte (e.g., a viral or cellular nucleic acid), a labelling agent (e.g., a reporter oligonucleotide attached thereto), or a product of an endogenous analyte and/or a labelling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some embodiments, the modified probe comprising one or more modified nucleotides generated as described herein may comprise one or more barcode sequences (e.g., on a first overhang of the primary probe).

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos). In some embodiment, any suitable probe for analyzing or detecting a barcode can be combined with the methods and reagents described herein such that the probe can be modified by attaching one or more modified nucleotides, such as cross-linkable nucleotides, once hybridized to the target nucleic acid.

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4′ complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and US 2021/0164039A1, which are hereby incorporated by reference in their entirety.

### III. Polynucleotides and Hybridization Complexes

In some aspects, the methods provided herein comprise use of a set of oligonucleotides, to generate a modified probe comprising one or more modified nucleotides. In some embodiments, the set of oligonucleotides comprises (i) a probe comprising a first overhang, a second overhang, and a hybridization region that hybridizes to a target nucleic acid, wherein the first overhang and second overhang do not hybridize to the target nucleic acid; and (ii) a first oligonucleotide, wherein the first oligonucleotide hybridizes to the second overhang. In some aspects, modified probe is used to analyze a target nucleic acid, e.g., messenger RNA in a cell or a biological sample. In some embodiments, the oligonucleotides comprise three different oligonucleotides, e.g., the probe, the first oligonucleotide, and an extension oligonucleotide comprising one or more modified nucleotides.

In some aspects, a target nucleic acid, a primary probe and a first oligonucleotide form a hybridization complex, wherein: the primary probe comprises a hybridization region that hybridizes to the target nucleic acid in the sample, a first overhang, and a second overhang, wherein the first and second overhangs do not hybridize to the target nucleic acid in the sample, and the second overhang hybridizes to the first oligonucleotide. In some embodiments, the first oligonucleotide (e.g., primer oligonucleotide) comprises one or more modified nucleotides.

In some aspects, a target nucleic acid, a primary probe, a first oligonucleotide, and a secondary probe form a hybridization complex, wherein: the primary probe comprises a hybridization region that hybridizes to the target nucleic acid in the sample, a first overhang, and a second overhang, wherein the first and second overhangs do not hybridize to the target nucleic acid in the sample, the second overhang hybridizes to the first oligonucleotide, and the secondary probe hybridizes to the first overhang, wherein the first overhang comprises one or more landing sequences capable of hybridizing to one or more secondary probes, optionally wherein the one or more landing sequences are barcode sequences. The hybridization complex may be subjected to one or more ligation steps, optionally to form a circular primary probe.

In some embodiments, the hybridization complex may be subjected to a ligation step, wherein the primary probe is ligated to an extension oligonucleotide, using the first oligonucleotide as a splint (e.g., splint oligonucleotide). In some embodiments, the extension oligonucleotide and the first oligonucleotide (e.g., splint oligonucleotide) each comprise one or more modified nucleotides. In some embodiments, the extension oligonucleotide comprises one or more modified nucleotides, and the first oligonucleotide (e.g., splint oligonucleotide) does not comprise a modified nucleotide. In some embodiments, the first oligonucleotide (e.g., splint oligonucleotide) comprises one or more modified nucleotides, and the extension oligonucleotide does not comprise a modified nucleotide. In some embodiments, one ligation step is needed for subsequent amplification to proceed. In some embodiments, the same splint oligonucleotide can be hybridized to multiple primary probes (e.g., via a common sequence shared by primary probes that bind different target nucleic acids). In some embodiments, different splint oligonucleotides can be hybridized to different primary probes. In some embodiments, a splint oligonucleotide may comprise one or more barcodes.

In some embodiments, one or more secondary probes are detectably labeled. In some embodiments, one or more secondary probes comprise one or more adaptor sequences that do not hybridize to the landing sequence(s) of the primary probes, wherein each adaptor sequence is capable of hybridizing to a detectably labeled oligonucleotide. In some aspects, the adaptor sequence is a region of an overhang of the secondary probe. In some examples, the adaptor sequence is complementary to a sequence comprised by a detectably labeled oligonucleotide. In some embodiments, the overhang of each secondary probe may comprise one or more adaptor sequences for hybridizing to one or more detectably labeled oligonucleotides (FIG. 5B).

In some aspects, provided herein is a probe comprising a first overhang, a second overhang, and a hybridization region for hybridizing to the target nucleic acid; and a first oligonucleotide that hybridizes to the second overhang. In some embodiments, the probe and first and/or second oligonucleotides are linear oligonucleotides (i.e., are not circular or circularized oligonucleotides).

In some embodiments, the first overhang of the probe is between or between about 5 and 40 nucleotides in length. In some embodiments, the first overhang is between or between about 5 and 15 nucleotides in length. In some embodiments, the first overhang is between or between about 15 and 20 nucleotides in length. In some embodiments, the first overhang is between or between about 20 and 25 nucleotides in length. In some embodiments, the first overhang is between or between about 25 and 30 nucleotides in length. In some embodiments, the first overhang is between or between about 30 and 35 nucleotides in length. In some embodiments, the first overhang is between or between about 25 and 30 nucleotides in length. In some embodiments, the first overhang is between or between about 35 and 40 nucleotides in length.

In some embodiments, the second overhang of the probe is between or between about 5 and 40 nucleotides in length. In some embodiments, the second overhang is between or between about 5 and 15 nucleotides in length. In some embodiments, the second overhang is between or between about 15 and 20 nucleotides in length. In some embodiments, the second overhang is between or between about 20 and 25 nucleotides in length. In some embodiments, the second overhang is between or between about 25 and 30 nucleotides in length. In some embodiments, the second overhang is between or between about 30 and 35 nucleotides in length. In some embodiments, the second overhang is between or between about 25 and 30 nucleotides in length. In some embodiments, the second overhang is between or between about 35 and 40 nucleotides in length.

In some embodiments, the first and/or second oligonucleotide is between or between about 5 and 40 nucleotides in length. In some embodiments, the first and/or second oligonucleotide is between or between about 5 and 15 nucleotides in length. In some embodiments, the first and/or second oligonucleotide is between or between about 15 and 20 nucleotides in length. In some embodiments, the first and/or second oligonucleotide is between or between about 20 and 25 nucleotides in length. In some embodiments, the first and/or second oligonucleotide is between or between about 25 and 30 nucleotides in length. In some embodiments, the first and/or second oligonucleotide is between or between about 30 and 35 nucleotides in length. In some embodiments, the first and/or second oligonucleotide is between or between about 25 and 30 nucleotides in length. In some embodiments, the first and/or second oligonucleotide is between or between about 35 and 40 nucleotides in length.

In some embodiments, the first and/or second oligonucleotide is blocked at the 3′ from extension, e.g., primer extension catalyzed by a polymerase. In some embodiments, the first and/or second oligonucleotide comprises a 3′ modification (e.g., a modification that blocks extension by a polymerase). Exemplary 3′ modifications include but are not limited to a 3′ ddC, 3′ inverted dT, a 3′ spacer phosphoramidite (e.g., a C3 spacer), 3′ amino, or a 3′ phosphorylation. In some embodiments, the probe and/or a modified probe comprising modified nucleotides incorporated in the extended overhang has a 5′-phosphate. In some embodiments, the first and/or second oligonucleotide has a 5′-phosphate. In some embodiments, the first and/or second extension oligonucleotide has a 5′-phosphate.

In some embodiments, the first and/or second oligonucleotide comprises a region that hybridizes to the end of the second overhang, and a region that does not hybridize to the second overhang. In some embodiments, the region that does not hybridize to the second overhang is used as a template for extension of the probe using a polymerase (e.g., to incorporate one or more modified nucleotides). In some embodiments, the region that does not hybridize to the second overhang comprises a region that hybridizes to an extension oligonucleotide. In some embodiments, the second overhang is ligated to the extension oligonucleotide using the first or second oligonucleotide as a splint (e.g., ligation with or without gap filling preceding ligation).

In some embodiments, the first and/or second extension oligonucleotide is between or between about 5 and 40 nucleotides in length. In some embodiments, the first and/or second extension oligonucleotide is between or between about 5 and 15 nucleotides in length. In some embodiments, the first and/or second extension oligonucleotide is between or between about 15 and 20 nucleotides in length. In some embodiments, the first and/or second extension oligonucleotide is between or between about 20 and 25 nucleotides in length. In some embodiments, the first and/or second extension oligonucleotide is between or between about 25 and 30 nucleotides in length. In some embodiments, the first and/or second extension oligonucleotide is between or between about 30 and 35 nucleotides in length. In some embodiments, the first and/or second extension oligonucleotide is between or between about 25 and 30 nucleotides in length. In some embodiments, the first and/or second extension oligonucleotide is between or between about 35 and 40 nucleotides in length.

In some embodiments, the first extension oligonucleotide comprise(s) a region that is capable of hybridizing to the first oligonucleotide (e.g., a region that is complementary to the first oligonucleotide). In some embodiments, the second extension oligonucleotide comprise(s) a region that is capable of hybridizing to the second oligonucleotide (e.g., a region that is complementary to the first oligonucleotide). In some embodiments, the first extension oligonucleotide comprises a region that does not hybridize to the first oligonucleotide (e.g., an overhang region). In some embodiments, the second extension oligonucleotide comprises a region that does not hybridize to the second oligonucleotide (e.g., an overhang region). In some embodiments, the extension region is used as a template for extension of the first or second oligonucleotide using a polymerase (e.g., extension to incorporate one or more modified nucleotides into the complement of the second overhang).

In some embodiments, the first and/or second extension oligonucleotide comprise(s) one or more, 2 or more, 3 or more, 4 or more, 5 or more, 6 or more, 7 or more, 8 or more, 9 or more, or 10 or more modified nucleotides. In some embodiments, the two or more modified nucleotides can comprise the same modifications or different modifications. In some embodiments, the two or more modified nucleotides can comprise different modifications having different functionalities (e.g., specific cross-linking or attachment to other agents vs. and non-specific cross-linking; or reversible cross-linking and irreversible cross-linking).

In some aspects, provided herein are one or more secondary probes capable of hybridizing to one or more regions of the first overhang, such as any of the detection oligonucleotides (e.g., detectably labelled oligonucleotides) or intermediate probes (e.g., secondary probes or higher order) described in Section VII.

The nucleic acid probes and/or probe sets disclosed herein can be introduced into a cell or used to otherwise contact a biological sample such as a tissue sample. The probes (e.g., the primary probes disclosed herein and/or any detectable probe disclosed herein, e.g., for FISH and/or RCA-based detection) may comprise any of a variety of entities that can hybridize to a nucleic acid, typically by Watson-Crick base pairing, such as DNA, RNA, LNA, PNA, etc. The nucleic acid probe may comprise a targeting sequence that is able to directly or indirectly bind to at least a portion of a target nucleic acid. The nucleic acid probe may be able to bind to a specific target nucleic acid (e.g., an mRNA, or other nucleic acids disclosed herein). In some embodiments, the nucleic acid probes may be detected using a detectable label, and/or by using secondary nucleic acid probes able to bind to the nucleic acid probes. In some embodiments, the nucleic acid probes (e.g., primary probes and/or secondary probes) are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease or exonuclease for cleavage or digestion).

Any probe disclosed herein, including primary nucleic acid probes, secondary nucleic acid probes, higher order nucleic acid probes, and detectably labeled nucleic acid probes, can be modified using methods disclosed herein.

In some embodiments, more than one type of primary nucleic acid probes may be contacted with a sample, e.g., simultaneously or sequentially in any suitable order, such as in sequential probe hybridization cycles. In some embodiments, more than one type of secondary nucleic acid probes may be contacted with a sample, e.g., simultaneously or sequentially in any suitable order, such as in sequential probe hybridization/unhybridization cycles. In some embodiments, the secondary probes may comprise probes that bind to a product of a primary probe targeting an analyte. In some embodiments, more than one type of higher order nucleic acid probes may be contacted with a sample, e.g., simultaneously or sequentially in any suitable order, such as in sequential probe hybridization/unhybridization cycles. In some embodiments, more than one type of detectably labeled nucleic acid probes (e.g., one or more primary detectable probes for smFISH readout and/or one or more secondary detectable probes for RCA readout) may be contacted with a sample, e.g., simultaneously or sequentially in any suitable order, such as in sequential probe hybridization/unhybridization cycles. In some embodiments, the detectably labeled nucleic acid probes can be used for smFISH readout and/or for RCA readout. In some embodiments, the detectably labeled probes (e.g., one or more primary detectable probes for smFISH readout and/or one or more secondary detectable probes for RCA readout) may comprise probes that bind to one or more primary probes, one or more secondary probes, one or more higher order probes, one or more intermediate probes between a primary/secondary/higher order probes, and/or one or more detectably or non-detectably labeled probes (e.g., as in the case of a hybridization chain reaction (HCR), a branched DNA reaction (bDNA), or the like). In some embodiments, at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable nucleic acid probes (e.g., primary, secondary, higher order probes, and/or detectably labeled probes) can be contacted with a sample, e.g., simultaneously or sequentially in any suitable order. Between any of the probe contacting steps disclosed herein, the method may comprise one or more intervening reactions and/or processing steps, such as modifications of a target nucleic acid, modifications of a probe or product thereof (e.g., via hybridization, ligation, extension, amplification, cleavage, digestion, branch migration, primer exchange reaction, click chemistry reaction, crosslinking, attachment of a detectable label, activating photo-reactive moieties, etc.), removal of a probe or product thereof (e.g., cleaving off a portion of a probe and/or unhybridizing the entire probe), signal modifications (e.g., quenching, masking, photo-bleaching, signal enhancement (e.g., via FRET), signal amplification, etc.), signal removal (e.g., cleaving off or permanently inactivating a detectable label), crosslinking, de-crosslinking, and/or signal detection.

The target-binding sequence (sometimes also referred to as the targeting region/sequence, the recognition region/sequence, or the hybridization region/sequence) of a probe may be positioned anywhere within the probe. For instance, the target-binding sequence of a primary probe that binds to a target nucleic acid can be 5′ or 3′ to any barcode sequence in the primary probe. Likewise, the target-binding sequence of a secondary probe (which binds to a primary probe or complement or product thereof) can be 5′ or 3′ to any barcode sequence in the secondary probe. In some embodiments, the target-binding sequence may comprise a sequence that is substantially complementary to a portion of a target nucleic acid. In some embodiments, the portions may be at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

The target-binding sequence of a primary nucleic acid probe may be determined with reference to a target nucleic acid (e.g., a cellular RNA or a reporter oligonucleotide of a labelling agent for a cellular analyte) that is present or suspected of being present in a sample. In some embodiments, more than one target-binding sequence can be used to identify a particular analyte comprising or associated with a target nucleic acid. The more than one target-binding sequence can be in the same probe or in different probes. For instance, multiple probes can be used, sequentially and/or simultaneously, that can bind to (e.g., hybridize to) different regions of the same target nucleic acid. In other examples, a probe may comprise target-binding sequences that can bind to different target nucleic acid sequences, e.g., various intron and/or exon sequences of the same gene (for detecting splice variants, for example), or sequences of different genes, e.g., for detecting a product that comprises the different target nucleic acid sequences, such as a genome rearrangement (e.g., inversion, transposition, translocation, insertion, deletion, duplication, and/or amplification).

After contacting the nucleic acid probes with a sample, the probes may be directly detected by determining detectable labels (if present), and/or detected by using one or more other probes that bind directly or indirectly to the probes or products thereof. The one or more other probes may comprise a detectable label. For instance, a primary nucleic acid probe can bind to a target nucleic acid in the sample, and a secondary nucleic acid probe can be introduced to bind to the primary nucleic acid probe, where the secondary nucleic acid probe or a product thereof can then be detected using detectable probes (e.g., detectably labeled probes). Higher order probes that directly or indirectly bind to the secondary nucleic acid probe or product thereof may also be used, and the higher order probes or products thereof can then be detected using detectably labeled probes.

In some instances, a secondary nucleic acid probe binds to a primary nucleic acid probe directly hybridized to the target nucleic acid. A secondary nucleic acid probe (e.g., a primary detectable probe or a secondary detectable probe disclosed herein) may contain a recognition sequence able to bind to or hybridize with a primary nucleic acid probe or a product thereof (e.g., an RCA product), e.g., at a barcode sequence or portion(s) thereof of the primary nucleic acid probe or product thereof. In some embodiments, a secondary nucleic acid probe may bind to a combination of barcode sequences (which may be continuous or spaced from one another) in a primary nucleic acid probe, a product thereof, or a combination of primary nucleic acid probes. In some embodiments, the binding is specific, or the binding may be such that a recognition sequence preferentially binds to or hybridizes with only one of the barcode sequences or complements thereof that are present. The secondary nucleic acid probe may also contain one or more detectable labels.

If more than one secondary nucleic acid probe is used, the detectable labels may be the same or different.

The recognition sequences may be of any length, and multiple recognition sequences in the same or different secondary nucleic acid probes may be of the same or different lengths. If more than one recognition sequence is used, the recognition sequences may independently have the same or different lengths. For instance, the recognition sequence may be at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the recognition sequence may be no more than 48, no more than 40, no more than 32, no more than 24, no more than 16, no more than 12, no more than 10, no more than 8, or no more than 6 nucleotides in length. Combinations of any of these are also possible, e.g., the recognition sequence may have a length of between 5 and 8, between 6 and 12, or between 7 and 15 nucleotides, etc. In some embodiments, the recognition sequence is of the same length as a barcode sequence or complement thereof of a primary nucleic acid probe or a product thereof. In some embodiments, the recognition sequence may be at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to the barcode sequence or complement thereof.

In some embodiments, a nucleic acid probe, such as a primary or a secondary nucleic acid probe, may also comprise 1, 2, 3, 4, 5, 6, 7, 8, 9, 10, 11, 12, 13, 14, 15 or more, 20 or more, 32 or more, 40 or more, or 50 or more barcode sequences. As an illustrative example, a first probe may contain a first target-binding sequence, a first barcode sequence, and a second barcode sequence, while a second, different probe may contain a second target-binding sequence (that is different from the first target-binding sequence in the first probe), the same first barcode sequence as in the first probe, but a third barcode sequence instead of the second barcode sequence. Such probes may thereby be distinguished by determining the various barcode sequence combinations present or associated with a given probe at a given location in a sample.

In some embodiments, the nucleic acid probes disclosed herein may be made using only 2 or only 3 of the 4 bases, such as leaving out all the “G”s and/or leaving out all of the

“C”s within the probe. Sequences lacking either “G”s or “C”s may form very little secondary structure, and can contribute to more uniform, faster hybridization in certain embodiments.

In some embodiments, a nucleic acid probe disclosed herein may contain a detectable label such as a fluorophore. In some embodiments, one or more probes of a plurality of nucleic acid probes used in an assay may lack a detectable label, while one or more other probes in the plurality each comprises a detectable label selected from a limited pool of distinct detectable labels (e.g., red, green, yellow, and blue fluorophores), and the absence of detectable label may be used as a separate “color.” As such, detectable labels are not required in all cases. In some embodiments, a primary nucleic acid probe disclosed herein lacks a detectable label. While a detectable label may be incorporated into an amplification product of a probe, such as via incorporation of a modified nucleotide into an RCA product of a circularized probe, the amplification product itself in some embodiments is not detectably labeled. In some embodiments, a probe that binds to the primary nucleic acid probe or a product thereof (e.g., a secondary nucleic acid probe that binds to a barcode sequence or complement thereof in the primary nucleic acid probe or product thereof) comprises a detectable label and may be used to detect the primary nucleic acid probe or product thereof. In some embodiments, a secondary nucleic acid probe disclosed herein lacks a detectable label, and a detectably labeled probe that binds to the secondary nucleic acid probe or a product thereof (e.g., at a barcode sequence or complement thereof in the secondary nucleic acid probe or product thereof) can be used to detect the second nucleic acid probe or product thereof. In some embodiments, signals associated with the detectably labeled probes (e.g., the first detectable probe which is detectably labelled, the second detectable probe which is detectably labelled, a detectably labeled probe that binds to the first detectable probe which itself is not detectably labelled, or a detectably labeled probe that binds to the second detectable probe which itself is not detectably labelled) can be used to detect one or more barcode sequences in the secondary probe and/or one or more barcode sequences in the primary probe, e.g., by using sequential hybridization of detectably labeled probes (e.g., smFISH-based detection), sequencing-by-ligation, and/or sequencing-by-hybridization. In some embodiments, the barcode sequences (e.g., in the secondary probe and/or in the primary probe) are used to combinatorially encode a plurality of analytes of interest. As such, signals associated with the detectably labeled probes at particular locations in a biological sample can be used to generate distinct signal signatures that each corresponds to an analyte in the sample, thereby identifying the analytes at the particular locations, e.g., for in situ spatial analysis of the sample.

In some embodiments, a nucleic acid probe herein comprises one or more other components, such as one or more primer binding sequences (e.g., to allow for enzymatic amplification of probes), enzyme recognition sequences (e.g., for endonuclease cleavage), or the like. The components of the nucleic acid probe may be arranged in any suitable order.

In some aspects, analytes are targeted by primary probes, which are barcoded through the incorporation of one or more barcode sequences (e.g., sequences that can be detected or otherwise “read”) that are separate from a sequence in a primary probe that directly or indirectly binds the targeted analyte. In some aspects, the primary probes are in turn targeted by secondary probes, which are also barcoded through the incorporation of one or more barcode sequences that are separate from a recognition sequence in a secondary probe that directly or indirectly binds a primary probe or a product thereof. In some embodiments, a secondary probe may bind to a barcode sequence in the primary probe. In some aspects, tertiary probes and optionally even higher order probes may be used to target the secondary probes, e.g., at a barcode sequence or complement thereof in a secondary probe or product thereof. In some embodiments, the tertiary probes and/or even higher order probes may comprise one or more barcode sequences and/or one or more detectable labels. In some embodiments, a tertiary probe is a detectably labeled probe that hybridizes to a barcode sequence (or complement thereof) of a secondary probe (or product thereof). In some embodiments, through the detection of signals associated with detectably labeled probes in a sample, the location of one or more analytes in the sample and the identity of the analyte(s) can be determined. In some embodiments, the presence/absence, absolute or relative abundance, an amount, a level, a concentration, an activity, and/or a relation with another analyte of a particular analyte can be analyzed in situ in the sample.

In some embodiments, provided herein are probes, probe sets, and assay methods to couple target nucleic acid detection, signal amplification (e.g., through nucleic acid amplification such as RCA, and/or hybridization of a plurality of detectably labeled probes, such as in hybridization chain reactions and the like), and decoding of the barcodes.

In some aspects, a primary probe, a secondary probe, and/or a higher order probe can be selected from the group consisting of a circular probe, a circularizable probe, and a linear probe. In some embodiments, a circular probe can be one that is pre-circularized prior to hybridization to a target nucleic acid and/or one or more other probes. In some embodiments, a circularizable probe can be one that can be circularized upon hybridization to a target nucleic acid and/or one or more other probes such as a splint. In some embodiments, a linear probe can be one that comprises a target recognition sequence and a sequence that does not hybridize to a target nucleic acid, such as a 5′ overhang, a 3′ overhang, and/or a linker or spacer (which may comprise a nucleic acid sequence or a non-nucleic acid moiety). In some embodiments, the sequence (e.g., the 5′ overhang, 3′ overhang, and/or linker or spacer) is non-hybridizing to the target nucleic acid but may hybridize to one another and/or one or more other probes, such as detectably labeled probes.

Specific probe designs can vary depending on the application. For instance, a primary probe, a secondary probe, and/or a higher order probe disclosed herein can comprise a circularizable probe that does not require gap filling to circularize upon hybridization to a template (e.g., a target nucleic acid and/or a probe such as a splint), a gapped circularizable probe (e.g., one that requires gap filling to circularize upon hybridization to a template), an L-shaped probe (e.g., one that comprises a target recognition sequence and a 5′ or 3′ overhang upon hybridization to a target nucleic acid or a probe), a U-shaped probe (e.g., one that comprises a target recognition sequence, a 5′ overhang, and a 3′ overhang upon hybridization to a target nucleic acid or a probe), a V-shaped probe (e.g., one that comprises at least two target recognition sequences and a linker or spacer between the target recognition sequences upon hybridization to a target nucleic acid or a probe), a probe or probe set for proximity ligation (such as those described in U.S. Pat. Nos. 7,914,987 and 8,580,504 incorporated herein by reference in their entireties, and probes for Proximity Ligation Assay (PLA) for the simultaneous detection and quantification of nucleic acid molecules and protein-protein interactions), or any suitable combination thereof. In some embodiments, a primary probe, a secondary probe, and/or a higher order probe disclosed herein can comprise a probe that is ligated to itself or another probe using DNA-templated and/or RNA-templated ligation. In some embodiments, a primary probe, a secondary probe, and/or a higher order probe disclosed herein can be a DNA molecule and can comprise one or more other types of nucleotides, modified nucleotides, and/or nucleotide analogues, such as one or more ribonucleotides. In some embodiments, the ligation can be a DNA ligation on a DNA template. In some embodiments, the ligation can be a DNA ligation on an RNA template, and the probes can comprise RNA-templated ligation probes. In some embodiments, a primary probe, a secondary probe, and/or a higher order probe disclosed herein can comprise a padlock-like probe or probe set, such as one described in US 2019/0055594, US 2021/0164039, US 2016/0108458, or US 2020/0224243, each of which is incorporated herein by reference in its entirety. Any suitable combination of the probe designs described herein can be used.

In some embodiments, a probe disclosed herein can comprise two or more parts. In some cases, a probe can comprise one or more features of and/or be modified based on: a split FISH probe or probe set described in WO 2021/167526A1 or Goh et al., “Highly specific multiplexed RNA imaging in tissues with split-FISH,” Nat Methods 17(7):689-693 (2020), which are incorporated herein by reference in their entireties; a Z-probe or probe set, such as one described in U.S. Pat. Nos. 7,709,198 B2, 8,604,182 B2, 8,951,726 B2, 8,658,361 B2, or Tripathi et al., “Z Probe, An Efficient Tool for Characterizing Long Non-Coding RNA in FFPE Tissues,” Noncoding RNA 4(3):20 (2018), which are incorporated herein by reference in their entireties; an HCR initiator or amplifier, such as one described in U.S. Pat. No. 7,632,641 B2, US 2017/0009278 A1, U.S. Pat. No. 10,450,599 B2, Dirks and Pierce, “Triggered amplification by hybridization chain reaction,” PNAS 101(43):15275-15278 (2004), Chemeris et al., “Real-time hybridization chain reaction,” Dokl. Biochem 419:53-55 (2008), Niu et al., “Fluorescence detection for DNA using hybridization chain reaction with enzyme-amplification,” Chem Commun (Camb) 46(18):3089-91 (2010), Choi et al., “Programmable in situ amplification for multiplexed imaging of mRNA expression,” Nat Biotechnol 28(11):1208-12 (2010), Song et al., “Hybridization chain reaction-based aptameric system for the highly selective and sensitive detection of protein,” Analyst 137(6):1396-401 (2012), Choi et al., “Third-generation in situ hybridization chain reaction: multiplexed, quantitative, sensitive, versatile, robust,” Development 145(12): dev165753 (2018), or Tsuneoka and Funato, “Modified in situ Hybridization Chain Reaction Using Short Hairpin DNAs,” Front Mol Neurosci 13:75 (2020), which are incorporated herein by reference in their entireties; a PLAYR probe or probe set, such as one described in US 2016/0108458 A1 or Frei et al., “Highly multiplexed simultaneous detection of RNAs and proteins in single cells,” Nat Methods 13(3):269-75 (2016), which are incorporated herein by reference in their entireties; a PLISH probe or probe set, such as one described in US 2020/0224243 A1 or Nagendran et al., “Automated cell-type classification in intact tissues by single-cell molecular profiling,” eLife 7:e30510 (2018), which are incorporated herein by reference in their entireties; a RollFISH probe or probe set such as one described in Wu et al., “RollFISH achieves robust quantification of single-molecule RNA biomarkers in paraffin-embedded tumor tissue samples,” Commun Biol 1, 209 (2018), which is hereby incorporated by reference in its entirety; a MERFISH probe or probe set, such as one described in WO 2020/123742 A1 (PCT/US2019/065857) or Chen et al., “Spatially resolved, highly multiplexed RNA profiling in single cells,” Science 348(6233):aaa6090 (2015), which are incorporated herein by reference in their entireties; or a primer exchange reaction (PER) probe or probe set, such as one described in US 2019/0106733 A1, which is hereby incorporated by reference in its entirety.

### IV. Ligation

In some aspects, provided herein are methods and compositions for performing a ligation that incorporates modified bases into a probe. In some embodiments, the ligation is performed in situ in a sample. In some embodiments, the primary probes are hybridized to a target nucleic acid in a sample. In some embodiments, the incorporation of modified bases into the primary probe is mediated by the ligation of an overhang of the primary probe to an extension oligonucleotide, which acts as a splint. In some embodiments, one or more modified nucleotides for crosslinking are attached to the 3′ end of the probe. In some embodiments, one or more modified nucleotides for crosslinking are attached to the 5′ end of the probe. In some embodiments, the methods provided herein involve ligating together of the 5′ overhang of the primary probe with an extension oligonucleotide. In some embodiments, the methods provided herein involve ligating together of the 3′ overhang of the primary probe with an extension oligonucleotide. In some aspects, the extension oligonucleotide comprises one or more modified nucleotides (such as any of the modified nucleotides described in Section VI), for anchoring or cross-linking of the modified probe to a scaffold. In some embodiments, an oligonucleotide is used as a splint oligonucleotide to mediate the ligation of the primary probe and the extension oligonucleotide comprising modified nucleotides, thereby modifying the primary probe hybridized to the target nucleic acid in the sample. In some embodiments, after ligation is performed, hybridized to the target nucleic acid in the sample is an extended primary probe with an extended second overhang that has modified bases incorporated.

In some embodiments, the ligation is performed under conditions permissive for specific hybridization of the oligonucleotides to one another. In some embodiments, the ligation of the primary probe and the extension oligonucleotide is performed under conditions permissive for specific hybridization of the primary probe to the splint oligonucleotide. In some embodiments, the ligation of the primary probe and the extension oligonucleotide is performed under conditions permissive for specific hybridization of the primary probe to the target nucleic acid. In some embodiments, the ligation is performed under conditions permissive for specific hybridization of the oligonucleotides to one another and/or to the target nucleic acid. In some embodiments, the ligation is a chemical ligation. In some embodiments, the chemical ligation involves click chemistry. In some embodiments, the ligation(s) of the primary probe involves enzymatic ligation.

In some embodiments, the enzymatic ligation involves use of a ligase. In some aspects, the ligase used herein comprises an enzyme that is commonly used to join oligonucleotides together. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 or T7 DNA ligase.

In some embodiments, the ligase is a ligase that has a DNA-splinted DNA ligase activity. In some embodiments, any or all of the splint oligonucleotide and primary probe are DNA molecules. In some embodiments, the splint oligonucleotide serves as a DNA template substrate for the ligation of the primary probe to the extension oligonucleotide.

In some embodiments, the primary probe and the extension oligonucleotide may be ligated directly or indirectly. “Direct ligation” means that the ends of the oligonucleotides hybridize immediately adjacently to one another to form a substrate for a ligase enzyme resulting in their ligation to each other. Alternatively, “indirect” means that the ends of the oligonucleotides hybridize non-adjacently to one another, i.e., separated by one or more intervening nucleotides or “gaps”. In some embodiments, primary probe and the extension oligonucleotide are not ligated directly to each other, but instead ligation occurs either via the intermediacy of one or more intervening (so-called “gap” or “gap-filling” (oligo)nucleotides) or by the extension of the 5′ or 3′ end of a primary probe to “fill” the “gap” corresponding to said intervening nucleotides (intermolecular ligation). In some cases, the gap of one or more nucleotides between the hybridized ends of the oligonucleotides may be “filled” by one or more “gap” (oligo)nucleotide(s) which are complementary to the splint oligonucleotide or primary probe. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between the primary probe and the extension oligonucleotide may be filled by a gap oligonucleotide or by extending the overhang of the primary probe. In some cases, ligation involves ligating the ends of the probe to at least one gap (oligo)nucleotide, such that the gap (oligo)nucleotide becomes incorporated into the resulting oligonucleotide (e.g., the ligated primary probe and extension oligonucleotide). In one aspect, the gap filling incorporates a modified nucleotide into the overhang of the primary probe. In other aspects, the gap filling incorporates two or more modified nucleotides into the overhang of the primary probe.

In some embodiments, the ligation of the primary probe and the extension oligonucleotide does not require gap filling. In other embodiments, the ligation the primary probe and the extension oligonucleotide is preceded by gap filling.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

### V. Extension/Amplification

In some embodiments, the methods of the invention comprise the step of extending one or more polynucleotides, such as the probe or a complement thereof, to incorporate one or more modified nucleotides. In some embodiments, the method comprises contacting a target nucleic acid with a probe and a first oligonucleotide to form a hybridization complex, e.g., using any of the primary probes and oligonucleotides (e.g., first or second oligonucleotides and optionally, extension oligonucleotides) described in Section III. In some embodiments, the second overhang is extended by a polymerase using the first oligonucleotide as a template. In some embodiments, the second overhang is ligated to an extension oligonucleotide using the first oligonucleotide as a splint, e.g., as performed using any of the exemplary methods described in Section IV. In some embodiments, a second oligonucleotide is then hybridized to the extended second overhang and used as a template for extension of the probe using a polymerase to incorporate one or more additional modified nucleotides. In some embodiments, after one or more rounds of extension is performed, hybridized to the target nucleic acid in the sample is an extended primary probe with an extended second overhang that has modified bases incorporated.

In some embodiments, the extension/amplification reaction is performed at a temperature lower than the melting temperature of the primary probe for hybridization to the target nucleic acid, the first oligonucleotide, and the secondary probe(s). In some aspects, the amplification steps can be performed at a temperature that is lower than the Tₘ of hybridization of the hybridization region between the primary probe oligonucleotide and target site on the target nucleic acid, at a temperature required for the amplification step. In some aspects, the amplification step is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors (including modified dNTPs and or dUTPs, such as any of the modified nucleotides described in Section VI) and other cofactors, the primary probe is elongated to incorporate one or more modified nucleotides. This extension/amplification step can utilize isothermal amplification or non-isothermal amplification. In some embodiments, the polymerase does not have a strand displacing activity, e.g., the polymerase is a T4 or T7 polymerase. This can prevent extension of the 3′ end of the first oligonucleotide from displacing the probe from the target nucleic acid. In some aspects, DNA polymerases that have been engineered or mutated to have desirable characteristics can be employed.

In some embodiments, the first oligonucleotide is blocked at the 3′ from extension, e.g., primer extension catalyzed by a polymerase. In some embodiments, the first oligonucleotide comprises a 3′ modification (e.g., a modification that blocks extension by a polymerase). Exemplary 3′ modifications include but are not limited to a 3′ ddC, 3′ inverted dT, a 3′ spacer phosphoramidite (e.g., a C3 spacer), 3′ amino, or a 3′ phosphorylation.

In some aspects, during the extension/amplification step, modified nucleotides can be added to the reaction to incorporate the modified nucleotides in the extension/amplification product (e.g., the extended probe or the extended oligonucleotide hybridized to the second overhang). In some aspects, the modified nucleotides can be employed, for example, for anchoring or cross-linking of the modified probe to a scaffold, to cellular structures and/or to other amplification products.

### VI. Crosslinkable Nucleotides and Crosslinking

In some embodiments, provided herein are methods and compositions for modifying a probe with one or more modified crosslinkable nucleotides, and performing crosslinking of modified nucleotides to the sample, a substrate and/or matrix. In some embodiments, the modified nucleotides have been attached (e.g., by extension with a polymerase or ligation) to a probe (e.g., a primary probe) that is hybridized to a target nucleic acid within a sample. In some embodiments, a generated modified probe (e.g., extended primary probe) is a linear oligonucleotide comprising from 5′ to 3′: a first overhang that can comprise one or more barcode sequences — a hybridization region that hybridizes to the target nucleic acid in the sample — an extended second overhang comprising one or more modified nucleotides. In some embodiments, a generated modified probe (e.g., extended primary probe) is a linear oligonucleotide comprising from 3′ to 5′: a first overhang can comprise one or more barcode sequences — a hybridization region that hybridizes to the target nucleic acid in the sample — an extended second overhang comprising one or more modified nucleotides.

In some embodiments, the one or more modified nucleotides comprise one or more crosslinkable nucleotides. In a non-limiting example, the one or more modified nucleotides comprise one or more cross-linkable nucleotides, e.g., photo-crosslinkable nucleotides such as UV-crosslinkable nucleotides. In some embodiments, the one or more modified nucleotides comprise a halogenated base, an azide-modified base, an amine-modified base, an aminoallyl-modified base, an octadiynyl dU, a thiol-modified base, a biotin-modified base, or a combination thereof. In some embodiments, the one or more modified nucleotides comprise nucleotides compatible with specific attachment to another molecule (e.g., attachment of a biotin-modified nucleotide to a labelling agent or analyte comprising a streptavidin label, or attachment, or attachment using click chemistry). In some embodiments, the one or more modified nucleotides comprise nucleotides capable of reversible crosslinking. For example, a thiol-modified base may form a disulfide bond with a thiol group, such that if the disulfide bond is broken (e.g., in the presence of a reducing agent), the cross-linked agent is released from the probe. In other cases, the modified base a reactive hydroxyl group that may be used for attachment. In some embodiments, the one or more modified nucleotides comprise at least one nucleotide that is internal after incorporation. In some embodiments, the one or more modified nucleotides comprise a 3′ or 5′ terminal nucleotide after incorporation.

In some aspects, the probe can be modified by attachment to one or more modified nucleotides, wherein the modified nucleotides are modified to incorporate a functional moiety (e.g., a functional moiety for attachment to the matrix). In some embodiments, the functional moiety can be a catalyst activated moiety. The functional moiety can be covalently cross-linked, copolymerize with or otherwise non-covalently bound to the matrix. In some embodiments, the functional moiety can react with a cross-linker. The functional moiety can be part of a ligand-ligand binding pair. dNTP or dUTP can be modified with the functional group, so that the function moiety is introduced into the DNA during amplification (e.g., during extension of the second overhang using the first and/or second oligonucleotide as a template, or extension of the complement of the second overhang using the second overhang as a template). Exemplary functional moieties of the modified nucleotides include an amine, acrydite, alkyne, aminoallyl, biotin, azide, and thiol. In the case of crosslinking, the functional moiety is cross-linked to modified dNTP or dUTP or both. Suitable exemplary cross-linker reactive groups include imidoester (DMP), succinimide ester (NETS), maleimide (Sulfo-SMCC), carbodiimide (DCC, EDC) and phenyl azide. In some embodiments, cross-linkers within the scope of the present disclosure may include a spacer moiety. Such spacer moieties may be functionalized. Such spacer moieties may be chemically stable. Such spacer moieties may be of sufficient length to allow amplification of the nucleic acid bound to the matrix. Suitable exemplary spacer moieties include polyethylene glycol, carbon spacers, photo-cleavable spacers and other spacers known to those of skill in the art and the like. In some embodiments, the modified nucleotides comprise modified dATP, dGTP, dCTP, and/or dTTP. In some embodiments, the modified nucleotides comprise modified dUTP (e.g., modified with aminoallyl, thiol, biotin, etc.). Suitable modified nucleotides are commercially available.

Exemplary modified nucleotides include amine-modified nucleotides. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification

In some embodiments, the methods provided herein comprise contacting the sample with one or more modified nucleotides and extending the second overhang using the first and/or second oligonucleotide as a template, or extending the complement of the second overhang using the second overhang as a template, wherein the extension incorporates one or more of the modified nucleotides into the second overhang or complement thereof.

In some embodiments, the methods provided herein further comprise crosslinking the one or more modified nucleotides to the sample, a substrate, and/or a matrix, e.g., a hydrogel matrix, thereby crosslinking the probe to the sample, the substrate, and/or the matrix, thereby increasing positional stability of the probe relative to the sample. In some embodiments, the one or more modified nucleotides are crosslinked to an endogenous molecule of the sample (e.g., an endogenous protein or nucleic acid). In some embodiments, the one or more modified nucleotides are crosslinked to an agent added to the sample, e.g., a labelling agent.

In some embodiments, crosslinking comprises contacting the sample with a crosslinking agent. In an example, the modified nucleotide is aminoallyl modified dNTP or dUTP, and the cross-linker is bis(succinimidyl)-nona-(ethylene glycol) or BS(PEG)9.

Biotin, or a derivative thereof, may also be used as a label on a nucleotide and/or a polynucleotide sequence, and subsequently bound by an avidin/streptavidin derivative (e.g., a streptavidin-conjugated protein), or an anti-biotin antibody or conjugate thereof. Digoxigenin may be incorporated as a label and subsequently bound by a detectably labeled anti-digoxigenin antibody (e.g., fluoresceinated anti-digoxigenin).

In some embodiments, crosslinking comprises exposing the sample to UV irradiation to activate a crosslinkable moiety (e.g., a photoactivatable crosslinking moiety).

In some aspects, the polynucleotides and/or amplification product (e.g., amplicon) can be anchored to a polymer matrix. For example, the polymer matrix can be a hydrogel. In some embodiments, one or more of the oligonucleotides probe(s) can be modified to contain functional groups that can be used as an anchoring site to attach the polynucleotide probes and/or amplification product to a polymer matrix.

Exemplary modifications and polymer matrix that can be employed in accordance with the provided embodiments comprise those described in, for example, US 2016/0024555, US 2018/0251833, US 2016/0024555, US 2018/0251833, US 2017/0219465, and US 2020/0071751, each of which is herein incorporated by reference in its entirety. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures. In some embodiments, the matrix comprises one or more types of functional moiety, wherein the functional moiety can react with the function moiety of the modified probe (e.g., extended probe with one or more modified bases incorporated), thereby immobilizing the probe. In some cases, a probe modified using a method provided herein with one or more modified bases incorporated may be tethered via a click reaction to a click reactive group functionalized hydrogel matrix (e.g., click gel). For example, the 5′azidomethyl-dUTP can be incorporated into probe and then immobilized to the hydrogel matrix functionalized with alkyne groups. Various click reactions may be used. In some embodiments, the tethering comprise providing conditions and buffer suitable for catalyzing the functional immobilization linkage between the modified probe and the matrix.

The modified probe (e.g., the extension or ligation product of the second overhang) may be immobilized within the matrix generally at the location of the target nucleic acid hybridized by the probe, thereby creating a localized probe and target nucleic acid complex. The probe may be immobilized within the matrix by covalent or noncovalent bonding, e.g., by crosslinking mediated by the one or more modified nucleotides. In this manner, the probe and target nucleic acid hybridized thereto may be considered to be attached to the matrix. By being immobilized to the matrix, such as by covalent bonding or cross-linking, the size and spatial relationship of the original target nucleic acids and probes is maintained. By being immobilized to the matrix, such as by covalent bonding or cross-linking, the target nucleic acids and probes are resistant to movement or unraveling under mechanical stress.

In some aspects, the modified probe and/or target nucleic acid hybridized thereto are copolymerized and/or covalently attached to the surrounding matrix thereby preserving their spatial relationship and any information inherent thereto. For example, if the probe is hybridized to a target nucleic acid within a cell embedded in the matrix, the modified probe can be crosslinked to the matrix, thereby preserving the spatial information of the target nucleic acid within the cell, thereby providing a subcellular localization distribution pattern. In some embodiments, the provided methods involve embedding the one or more polynucleotide probe sets and target nucleic acids in the presence of hydrogel subunits to form one or more hydrogel-embedded probe-target nucleic acid hybridization complexes. In some embodiments, the hydrogel-tissue chemistry described comprises covalently attaching nucleic acids (e.g., any of the modified probes described herein) to in situ synthesized hydrogel for tissue clearing, enzyme diffusion, and multiple-cycle sequencing while an existing hydrogel-tissue chemistry method cannot. In some embodiments, the one or more modified nucleotides can comprise one or more amine-modified nucleotides that can be functionalized with an acrylamide moiety using acrylic acid N-hydroxysuccinimide esters, and copolymerized with acrylamide monomers to form a hydrogel. In some embodiments, the provided methods involve crosslinking the one or more polynucleotides (e.g., generated modified probes comprising one or more modified nucleotides) in the presence of hydrogel subunits prior to clearing treatments (e.g., SDS or Proteinase K).

### VII. Detection and Analysis

In some aspects, the provided methods involve analyzing, e.g., detecting or determining, one or more sequences present in the target nucleic acid and/or in the probes modified by the methods described herein. In some embodiments, the detecting comprises hybridizing one or more detectably labeled probes to the probe (e.g., via hybridization to landing regions on the first overhang of the probe, or via hybridization to secondary probes (or other intermediate probes) that hybridize to the landing regions on the first overhang of the probe). In some embodiments, the analysis comprises determining the sequence of all or a portion of the first overhang of the probe, (e.g., a barcode sequence), wherein the sequence is indicative of a sequence of the target nucleic acid. In some embodiments, a detectable labeled probe, any intermediate probes, and/or the barcode sequence of the primary probe can be associated with the identity of the target nucleic acid.

In some embodiments, the methods comprise sequencing or detecting all or a portion of the first overhang, such as one or more barcode sequences present in the first overhang of the probe. In some embodiments, the sequence of the first overhang is indicative of a sequence of the target nucleic acid to which the probe is hybridized. In some embodiments, the analysis and/or sequence determination comprises sequencing all or a portion of the first overhang and/or in situ hybridization to the first overhang. In some embodiments, the sequencing step involves sequencing by hybridization, sequencing by ligation, sequencing by synthesis, sequencing by binding, and/or fluorescent in situ sequencing (FISSEQ), hybridization-based in situ sequencing and/or wherein the in situ hybridization comprises sequential fluorescent in situ hybridization. In some instances, detecting of sequences in the first overhang, such as one or more barcode sequences present in the first overhang of the probe, can be performed using barcoding schemes and/or barcode detection schemes as described in single-molecule fluorescent in situ hybridization (smFISH), multiplexed error-robust fluorescence in situ hybridization (MERFISH) or sequential fluorescence in situ hybridization (seqFISH+). In any of the preceding implementations, the methods provided herein can include analyzing the barcodes by sequential hybridization and detection with a plurality of labelled probes (e.g., detectably labeled oligonucleotides).

In some embodiments, the analysis and/or sequence determination comprises detecting a polymer generated by a chain reaction of hybridization of multiple detectably labelled oligonucleotides (e.g., a hybridization chain reaction (HCR) reaction), see e.g., US2017/0009278, which is incorporated herein by reference, for exemplary probes and HCR reaction components. In some embodiments, each primary probe can be hybridized by more than one detectably labeled oligonucleotide, thereby allowing signal amplification. In some embodiments, each secondary probe can be hybridized by more than one detectably labeled oligonucleotide, thereby allowing signal amplification. In some embodiments, the detection or determination comprises hybridizing to the first overhang a detection oligonucleotide labeled with a fluorophore, an isotope, a mass tag, or a combination thereof. In some embodiments, the detection or determination comprises imaging the probe hybridized to the target nucleic acid (e.g., imaging one or more detectably labeled probes hybridized thereto). In some embodiments, the target nucleic acid is an mRNA in a tissue sample, and the detection or determination is performed when the target nucleic acid and/or the amplification product is in situ in the tissue sample. In some embodiments, the target nucleic acid is an amplification product (e.g., a rolling circle amplification product).

In some aspects, the provided methods comprise imaging the probe hybridized to the target nucleic acid, for example, via binding of the detectably labeled oligonucleotide and detecting the detectable label. In some embodiments, the detectably labeled oligonucleotide comprises a detectable label that can be measured and quantitated. The terms “label” and “detectable label” comprise a directly or indirectly detectable moiety that is associated with (e.g., conjugated to) a molecule to be detected, e.g., a probe that is a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a secondary probe that is a detectably labeled oligonucleotide containing a detectable label can be used to detect one or more probe(s) (e.g., modified and/or crosslinked probes) described herein. In some embodiments, a detectably labeled oligonucleotide hybridizes to an unlabeled intermediate probe (e.g., secondary probe) that hybridizes to the primary probe (e.g., modified and/or crosslinked probe) described herein. In some embodiments, the methods involve incubating the detectably labeled oligonucleotide containing the detectable label with the sample, washing unbound detectably labeled oligonucleotides, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991), each of which is herein incorporated by reference in its entirety. In some embodiments, exemplary techniques and methods methodologies applicable to the provided embodiments comprise those described in, for example, U.S. Pat. Nos. 4,757,141, 5,151,507 and 5,091,519, each of which is herein incorporated by reference in its entirety. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and US 5,688,648 (energy transfer dyes) , each of which is herein incorporated by reference in its entirety. Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, each of which is herein incorporated by reference in its entirety. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6xHis), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and 5,192,782, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe (e.g., detectably labeled oligonucleotide). In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe (e.g., detectably labeled oligonucleotide). Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing can be performed in situ. In some embodiments, sequencing in situ can be performed on one or more barcodes on the first overhang of the modified probe. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (i.e., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932, all of which are herein incorporated by reference in their entireties. Exemplary techniques for in situ sequencing comprise, but are not limited to, STARmap (described for example in Wang et al., (2018) Science, 361(6499) 5691, herein incorporated by reference in its entirety), MERFISH (described for example in Moffitt, (2016) Methods in Enzymology, 572, 1-49, herein incorporated by reference in its entirety), hybridization-based in situ sequencing (HybISS) (described for example in Gyllborg et al., Nucleic Acids Res (2020) 48(19):e112, herein incorporated by reference in its entirety), and FISSEQ (described for example in US 2019/0032121, which is herein incorporated by reference in its entirety).

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing can be performed by sequential fluorescence hybridization (e.g., sequencing by hybridization). Sequential fluorescence hybridization can involve sequential hybridization of detection probes (e.g., detectably labeled oligonucleotides) comprising an oligonucleotide and a detectable label.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, the barcodes of the primary or secondary probes are targeted by detectably labeled oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detectably labeled oligonucleotides). Exemplary decoding schemes are described in Eng et al., “Transcriptome-scale Super-Resolved Imaging in Tissues by RNA SeqFISH+,” Nature 568(7751):235-239 (2019); Chen et al.,“Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; US 2021/0017587 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence (e.g., on the first overhang of the modified probe). Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), each of which is herein incorporated by reference in its entirety.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, each of which is herein incorporated by reference in its entirety.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### VIII. Compositions, Kits, and Systems

In some embodiments, disclosed herein is a composition that comprises a complex containing a target nucleic acid, a probe, and a first oligonucleotide, e.g., any of the target nucleic acids, probes, and first oligonucleotides described in Section III. In some embodiments, the complex further comprises an extension oligonucleotide and/or secondary probe, e.g., as described in Section III and any detectably labeled oligonucleotides, e.g., as described in Section VII. In some embodiments, the first oligonucleotide and/or the extension oligonucleotide comprise modified nucleotides, such that the modified nucleotides are attached to the primary probe. In some embodiments, the composition further comprises one or more modified nucleotides, e.g., any of the modified nucleotides described in Section VI.

In some embodiments, disclosed herein is a composition that comprises an extension or ligation product of the probe (e.g., an extended probe with an extended second overhang), wherein the extension or ligation product comprises one or more (e.g., two or more) modified nucleotides. In some embodiments, the extension product is formed using the first oligonucleotide as a template, and thus comprises a sequence complementary to the first oligonucleotide. In some embodiments, the ligation product is formed using the first oligonucleotide as a splint. In some embodiments, disclosed herein is a composition that comprises a product of a first ligation of the probe to a first extension oligonucleotide, followed by (i) a second ligation of the ligation product to a second extension oligonucleotide using a second oligonucleotide as a splint, or (ii) extension of the ligation product using the second oligonucleotide as a template, wherein the extension comprises incorporation of one or more modified nucleotides. In some embodiments, the first and second extension oligonucleotides can comprise one or more modified nucleotides. In some embodiments, the first and second extension oligonucleotides can be the same or different.

Also provided herein are kits, for example comprising one or more oligonucleotides, e.g., any described in Section III, and instructions for performing the methods provided herein. In some embodiments, the kits further comprise one or more reagents for performing the methods provided herein (e.g., one or more modified nucleotides, such as any of the modified nucleotides described in Section VI). In some embodiments, the kits further comprise one or more reagents required for one or more steps comprising hybridization, ligation, extension, detection, sequencing, and/or sample preparation as described herein. In some embodiments, the kit further comprises a target nucleic acid, e.g., any described in Section III. In some embodiments, the kit further comprises any intermediate probes and detectably labeled oligonucleotides, e.g., as described in Section VII. In some embodiments, any or all of the oligonucleotides are DNA molecules. In some embodiments, the target nucleic acid is a messenger RNA molecule. In some embodiments, the target nucleic acid is a probe (e.g., a padlock probe) or an amplification product thereof (e.g., a rolling circle amplification product). In some embodiments, the kit further comprises a ligase, for instance for forming a ligated, modified probe from the probe and the extension oligonucleotide, using the first oligonucleotide as a splint. In some embodiments, the ligase has DNA-splinted DNA ligase activity. In some embodiments, the kit further comprises a polymerase, for instance for performing extension of the probe to attach modified nucleotides. In some embodiments, the polymerase is capable of using the second overhang of the probe as primer and first or second oligonucleotide as a template for extension to incorporate one or more modified nucleotides, e.g., using any of the methods described in Section V. In some embodiments, the polymerase is capable of using the first oligonucleotide as primer and the probe as a template for extension to incorporate one or more modified nucleotides, e.g., using any of the methods described in Section V. In some embodiments, the kits may contain reagents for forming a functionalized matrix (e.g., a hydrogel), such as any suitable functional moieties. In some examples, also provided are buffers and reagents for tethering the modified probes to the functionalized matrix. The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as detectably labeled oligonucleotides or detectable labels. In some embodiments, the kits optionally contain other components, for example nucleic acid primers, enzymes and reagents, buffers, nucleotides, modified nucleotides, reagents for additional assays.

IX. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to identify or detect single nucleotides of interest in target nucleic acids. In some aspects, the provided embodiments can be used to crosslink the primary probes via modified nucleotides, e.g., to a matrix, to increase the stability of the primary probe in situ.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### X. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, i.e., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the T. for the specific sequence at a defined ionic strength and pH. The melting temperature T. can be the temperature at which a population of double-stranded nucleic acid molecules becomes half dissociated into single strands. Several equations for calculating the T. of nucleic acids are well known in the art. As indicated by standard references, a simple estimate of the T. value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)).

Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5× SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, i.e. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeg™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif), sequencing by ion detection technologies (such as Ion Torrent™ technology, Life Technologies, Carlsbad, Calif); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods. “Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLE

### Example 1

### Modification of Probes, Crosslinking, and Detection

This Example describes various exemplary methods of modifying a probe with one or more crosslinkable nucleotides.

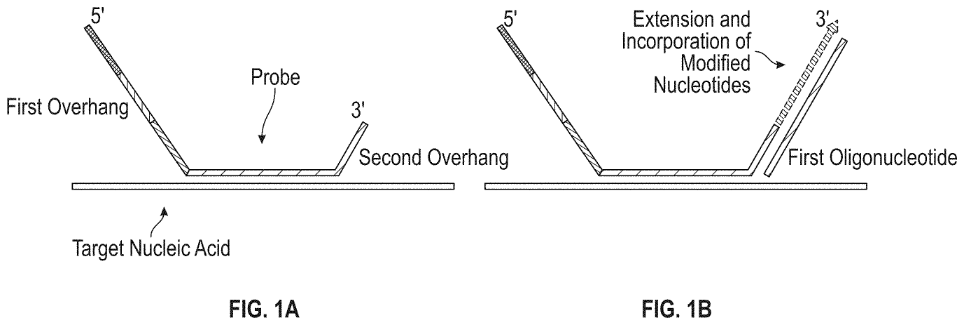

In an example, a probe and a first oligonucleotide are contacted with a target nucleic acid in a sample under conditions permitting hybridization of a hybridization region of the probe to the target nucleic acid, and hybridization of the first oligonucleotide to a second overhang of the probe. As shown in FIG. 1A, the probe comprises (i) a hybridization region that hybridizes to the target nucleic acid in the sample, (ii) a first overhang, and (iii) a second overhang, wherein the first and second overhangs do not hybridize to the target nucleic acid. In some embodiments, the second overhang can be at the 3′ end of the probe, as shown.

The sample can be contacted with the probe and the first oligonucleotide simultaneously, or the sample can be contacted first with the probe and then with the first oligonucleotide, or first with the first oligonucleotide and then with the probe. In some cases, one or more washes can be performed to remove unbound probes.

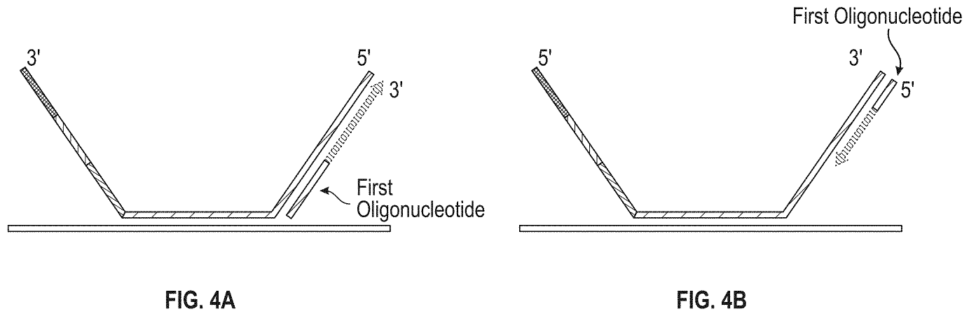

The sample with probes hybridized is then contacted with a polymerase (e.g., a T4 or T7 polymerase) and a mixture of nucleotides comprising one or more modified nucleotides (e.g., comprising a halogenated base, an azide-modified base, an amine-modified base, an aminoallyl-modified base, an octadiynyl dU, a thiol-modified base, a biotin-modified base, or a combination thereof), and incubated under conditions suitable for extension of the second overhang of the probe or of the first oligonucleotide using the polymerase. In one example, the first oligonucleotide hybridizes to the second overhang, providing a template for extension of the probe using a polymerase to incorporate one or more modified nucleotides, and using the first oligonucleotide as a template (FIG. 1B). In another example as shown in FIGS. 4A-4B, the one or more modified nucleotides are incorporated into a complement of the second overhang using a first oligonucleotide as a primer and the second overhang as a template for extension by a polymerase. In this example, the modified oligonucleotides are indirectly attached to the probe by hybridization of the modified extended first oligonucleotide and the second overhang. In some examples as shown in the figure, the second overhang is at the 5′ end of the probe and the first oligonucleotide hybridizes at the 3′ end of the second overhang (FIG. 4A). In other examples, the second overhang is at the 3′ end of the probe and the first oligonucleotide hybridizes at the 3′ end of the second overhang (FIG. 4B).

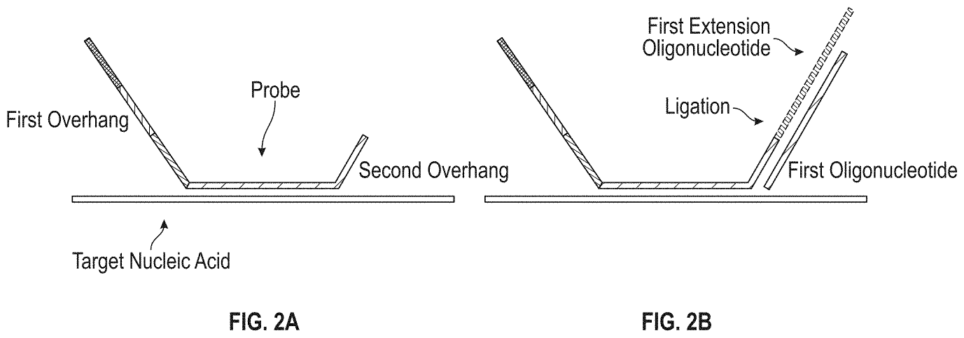

In another example, the method further comprises contacting the sample with a first extension oligonucleotide comprising one or more modified nucleotides, such as any of the modified nucleotides described above. The first extension oligonucleotide can be added to the sample simultaneously with the probe and/or the first oligonucleotide, or can be added before or after the probe and/or first oligonucleotide. The sample can be contacted with a ligase (e.g., T4 DNA ligase). The extension oligonucleotide can hybridize to the first oligonucleotide, and the first oligonucleotide can act as a splint for ligation of the first extension oligonucleotide to the second overhang. FIGS. 2A-2B show an exemplary method of modifying a probe by attaching an extension oligonucleotide comprising one or more modified nucleotides to the second overhang by ligation, wherein the first oligonucleotide acts as a splint to template the ligation. As shown in FIG. 2A, the second overhang can be located at either the 5′ end or the 3′ end of the probe. The sample is contacted with a first extension oligonucleotide comprising one or more modified nucleotides and a first oligonucleotide, wherein the first oligonucleotide hybridizes to the second overhang. In some embodiments, the first extension oligonucleotide can extend beyond the first oligonucleotide (i.e., can comprise a region that does not hybridize to the first oligonucleotide), as shown in FIG. 2B.

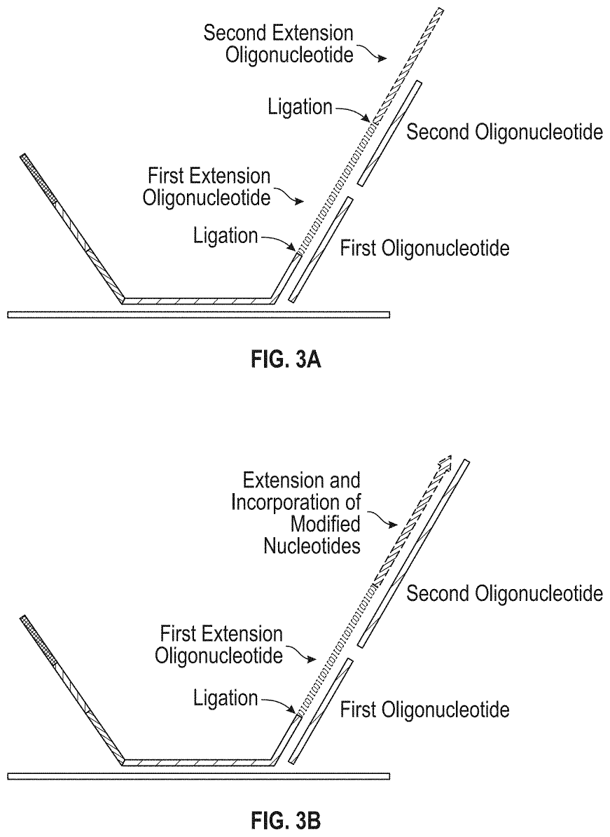

FIG. 3A shows an exemplary method of modifying a probe by attaching a first and a second extension oligonucleotide, wherein the first extension oligonucleotide is ligated to the second overhang using a first oligonucleotide as a splint (i.e., as a template for ligation), and the second extension oligonucleotide is ligated to the ligation product of the second overhang using a second oligonucleotide as a splint. The first and/or the second extension oligonucleotide can comprise one or more modified nucleotides. FIG. 3B shows an exemplary method of modifying a probe by attaching a first extension oligonucleotide to the second overhang by ligation using a first oligonucleotide as a splint, and extending the ligation product of the second overhang using a second oligonucleotide as a template. The second oligonucleotide hybridizes to the extended second overhang, providing a template for extension of the probe using a polymerase to incorporate one or more modified nucleotides.

In some cases, after the probe has been modified either by extension and/or ligation to attach one or more modified nucleotides to the overhang, the method then comprises crosslinking the one or more modified nucleotides of the modified probe (e.g., the ligation or extension product of the probe) to a matrix. The crosslinking can comprise contacting the sample with a crosslinking agent. In an example, the modified nucleotide is aminoallyl modified dNTP or dUTP, and the cross-linker is bis(succinimidyl)-nona-(ethylene glycol) or BS(PEG)9. In another example, the one or more modified nucleotides can comprise one or more amine-modified nucleotides that can be functionalized with an acrylamide moiety using acrylic acid N-hydroxysuccinimide esters, and copolymerized with acrylamide monomers to form a hydrogel. In some cases, optional tissue treatment steps may be performed after the crosslinking, such as tissue clearing.

FIG. 5A shows an exemplary method wherein the one or more modified nucleotides comprise one or more cross-linkable nucleotides. Cross-linking is indicated by an “x”. In some embodiments, the methods provided herein allow incorporation of multiple crosslinkable nucleotides into the probe. In some embodiments, the method comprises crosslinking the one or more modified nucleotides to the sample, a substrate, and/or a matrix, e.g., a hydrogel matrix, thereby crosslinking the probe to the sample, the substrate, and/or the matrix, thereby increasing positional stability of the probe relative to the sample. Ins some embodiments, the probe is crosslinked to an endogenous molecule of the sample, e.g., an endogenous protein.

FIG. 5B shows an exemplary method of detecting a modified probe by hybridization of one or more secondary probes to the first overhang of the probe. In some embodiments, the first overhang can comprise one or more barcode sequences. In some embodiments, the first overhang can comprise one or more landing sequences capable of hybridizing to one or more secondary probes, optionally wherein the one or more landing sequences are barcode sequences. The one or more secondary probes can be detectably labeled, or can comprise one or more adaptor sequences that do not hybridize to the landing sequence(s), wherein each adaptor sequence is capable of hybridizing to one or more detectably labeled oligonucleotides, as shown in FIG. 5B. It will be understood that the detection methods are not limited to the example shown, and that any suitable method can be used to detect the probe, including for example sequential hybridization, sequencing by hybridization, sequencing by ligation, sequencing by synthesis, sequencing by binding, hybridization chain reaction, or any combination thereof.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1-73. (canceled)

74. A method of modifying a probe, comprising:
(a) contacting a probe, a first oligonucleotide, and a sample comprising a target nucleic acid in any suitable order, wherein:
the probe comprises (i) a hybridization region that hybridizes to the target nucleic acid in the sample, (ii) a first overhang, and (iii) a second overhang, wherein the first and second overhangs do not hybridize to the target nucleic acid, and
the second overhang hybridizes to the first oligonucleotide; and
(b) attaching one or more modified nucleotides to the second overhang using the first oligonucleotide as a template or to a complement of the second overhang using the first oligonucleotide as a primer, thereby modifying the probe hybridized to the target nucleic acid in the sample.

75. The method of claim 74, wherein the second overhang is at the 3′ of the probe, and wherein (i) a polymerase catalyzes extension of the second overhang using the first oligonucleotide as a template, thereby attaching the one or more modified nucleotides to the second overhang, or (ii) the attaching step comprises ligating the second overhang and a first extension oligonucleotide using the first oligonucleotide as a splint.

76. The method of claim 74, wherein the first oligonucleotide is blocked at the 3′ from extension and/or wherein the first oligonucleotide comprises a 3′ modification.

77. The method of claim 74, wherein the first oligonucleotide comprises a 3′ modification selected from the group consisting of 3′ ddC, 3′ inverted dT, a 3′ spacer phosphoramidite, 3′ amino, or a 3′ phosphorylation.

78. The method of claim 74, wherein the second overhang is at the 5′ of the probe, and wherein the attaching step comprises ligating the second overhang and a first extension oligonucleotide using the first oligonucleotide as a splint.

79. The method of claim 74, wherein the method further comprises contacting the sample with a second oligonucleotide, wherein the second oligonucleotide hybridizes to the second overhang of the modified probe.

80. The method of claim 79, wherein the method comprises a step (c) of attaching one or more modified nucleotides to the second overhang of the modified probe using the second oligonucleotide as a template or into a complement of the ligation product of the second overhang using the second oligonucleotide as a primer, thereby further modifying the probe hybridized to the target nucleic acid in the sample.

81. The method of claim 74, wherein the one or more modified nucleotides comprise one or more cross-linkable nucleotides and/or wherein the one or more modified nucleotides comprise a halogenated base, an azide-modified base, an octadiynyl dU, a thiol-modified base, a biotin-modified base, or a combination thereof.

82. The method of claim 81, further comprising crosslinking the one or more modified nucleotides to the sample, a substrate, and/or a matrix.

83. The method of claim 74, wherein the one or more modified nucleotides comprise at least one nucleotide that is internal after incorporation.

84. The method of claim 74, wherein the first overhang comprises one or more barcode sequences.

85. The method of claim 74, wherein the first overhang comprises one or more landing sequences capable of hybridizing to one or more secondary probes.

86. The method of claim 85, wherein the one or more secondary probes are detectably labeled.

87. The method of claim 74, wherein the sample is a tissue sample.

88. The method of claim 74, wherein the method further comprises analyzing localization of the target nucleic acid in the sample.

89. The method of claim 74, wherein the method further comprises detecting a signal indicative of the probe hybridized to the target nucleic acid in the sample.

90. The method of claim 74, wherein the attaching step is performed after contacting the sample comprising the target nucleic acid with the probe and the first oligonucleotide.

91. The method of claim 74, wherein the attaching step is performed after the probe is hybridized to the target nucleic acid.

92. A method of modifying a probe, comprising:
(a) contacting a probe, a first oligonucleotide, and a sample comprising a target nucleic acid in any suitable order, wherein:
the probe comprises (i) a hybridization region that hybridizes to the target nucleic acid in the sample, (ii) a first overhang, and (iii) a second overhang, wherein the first and second overhangs do not hybridize to the target nucleic acid, and
the second overhang hybridizes to the first oligonucleotide; and
(b) ligating the second overhang to a first extension oligonucleotide comprising one or more modified nucleotides, using the first oligonucleotide as a template, thereby modifying the probe hybridized to the target nucleic acid in the sample.

93. A method of modifying a probe, comprising:
(a) contacting a probe, a first oligonucleotide, and a sample comprising a target nucleic acid in any suitable order, wherein:
the probe comprises (i) a hybridization region that hybridizes to the target nucleic acid in the sample, (ii) a first overhang, and (iii) a second overhang at the 3′ end of the probe, wherein the first and second overhangs do not hybridize to the target nucleic acid, and
the second overhang hybridizes to the first oligonucleotide; and
(b) extending the second overhang using a polymerase to incorporate one or more modified nucleotides to the second overhang using the first oligonucleotide as a template, thereby modifying the probe hybridized to the target nucleic acid in the sample;
wherein the first oligonucleotide is a linear oligonucleotide.
